(19) United States
(12) Patent Application Publication
(10) Pub. No.: US 2009/0130118 A1
(43) Pub. Date: May 21, 2009

(54) SCUTELLARIA BARBATA EXTRACT AND COMBINATIONS FOR THE TREATMENT OF CANCER

(75) Inventor: Isaac Cohen, Piedmont, CA (US)

Correspondence Address: WILSON SONSINI GOODRICH & ROSATI, 650 PAGE MILL ROAD, PALO ALTO, CA, 94304-1050, US

(73) Assignee: BIONOVO, INC., Emeryville, CA (US)

(21) Appl. No.: 12/274,264

(22) Filed: Nov. 19, 2008

Related U.S. Application Data
(60) Provisional application No. 60/989,072, filed on Nov. 19, 2007.

(51) Int. Cl. A61K 39/395; A61K 36/539; A61P 35/00

(52) U.S. Cl. 424/141.1; 424/741

(57) ABSTRACT

An extract of Scutellaria barbata D. Don is effective in the arrest of cancer cell growth in the G1 phase, the induction of apoptosis in cancer cells and the shrinking of solid cancers. The extract may be prepared as a pharmaceutical composition for administration to mammals for the treatment of solid cancers, such as epithelial cancers. Such epithelial cancers include breast cancer and ovarian cancers. The extract is obtained from Scutellaria barbata D. Don by contacting aerial portions of a plant from the species Scutellaria barbata D. Don with an aqueous or alcoholic solvent.

This application claims benefit of priority under 35 U.S.C. §119(e) from provisional patent application No. 60/989,072, filed Nov. 19, 2007, which is incorporated herein by reference in its entirety.

### BACKGROUND OF THE INVENTION

While advances in early detection and adjuvant therapy for breast cancer have had a favorable impact on patient survival in general, patients who develop advanced metastatic breast cancer are generally likely to face a less favorable prognosis. Commonly used hormonal and chemotherapeutic agents can lead to transient regression of tumors and can also palliate symptoms related to cancer. However, these treatments are often accompanied by toxicities and intolerable side effects and eventually become ineffective in controlling advanced stage breast cancer and its symptoms. Improvements in survival are modest, even with newer targeted biological agents. Moreover, in most metastatic cancers resistance to available conventional treatment ultimately develops or excessive side effects are seen with conventional therapies.

It is interesting to note that greater than 60% of all chemotherapeutic agents used in the treatment of breast cancer are derived from natural substances (Newman 2003). A fairly recent example is the development of taxanes from the Pacific yew tree, Taxus brevifolia. Throughout the world, it is estimated that approximately 80% of the world population still relies on botanical medicine as the primary source of therapy. In the West, botanical medicine is considered a popular form of complementary and alternative medicine among patients diagnosed with cancer. However, few clinical trials have been conducted to firmly assess the safety and efficacy of botanical agents for the treatment of breast cancer, despite anecdotal case reports of cures and clinical efficacy in women who have relied solely on botanical medicine for treatment. It has previously been shown that the aqueous extract of Scutellaria Barbata can lead to growth inhibition of breast cancer cell lines in vitro (“Antiproliferative activity of Chinese medicinal herbs on breast cancer cells in vitro,” Anticancer Res., 22(6C):3843-52 (2002)). BZL101, a concentrated aqueous extract of Scutellaria Barbata, was evaluated for antiproliferative activity on five breast cancer cell lines (SK-BR-3, MCF7, MDA-MB-231, BT-474, and MCNeuA). These cell lines represent important prognostic phenotypes of breast cancer expressing a range of estrogen and HER2 receptors. BZL101, tested at a 1:10 dilution (15μg/ml), demonstrated >50% growth inhibition on four of the five cell lines (Campbell, 2002). BZL101 showed >50% growth inhibition on a panel of lung, prostate and pancreatic cancer cell lines. BZL101 at the same dose did not cause >25% of growth inhibition on normal human mammary cells (HuMEC), demonstrating selectivity to cancer cells (Table 1). More so, BZL101 had a mild mitogenic effect on normal human lymphocytes. In cell cycle analysis, BZL101 caused an S phase burst and G1 arrest. BZL101 also attenuated mitochondrial membrane potential causing caspase-independent high molecular grade (HMG) apoptosis.

There is a need for therapies for treatment of patients having metastatic cancers. There is also a need for therapies with reduced, and more specifically minimal, toxicity for patients having metastatic cancers. In particular, there is a need for novel therapies with relatively low toxicity for the treatment of metastatic solid tumors, such as epithelial tumors, and more particularly breast and ovarian cancers.

These and other needs are met by embodiments of the invention.

### SUMMARY OF THE INVENTION

The foregoing and further needs are met by embodiments of the invention, which provide methods for the treatment of cancer.

In some embodiments, the invention comprises a method of treating cancer in a patient, comprising administering to the patient a therapeutically effective amount of an extract of Scutellaria barbata D. Don and at least one additional agent, which inhibits aromatase activity. In some embodiments, the invention comprises a method of treating cancer in a patient, comprising administering to the patient a therapeutically effective amount of an extract of Scutellaria barbata D. Don and at least one additional agent, which antagonizes androgen activity.

In some embodiments, the invention comprises a method of treating cancer in a patient, comprising administering to the patient a therapeutically effective amount of an extract of Scutellaria barbata D. Don and at least one additional agent, which agonizes gonadotropin releasing hormone activity.

In some embodiments, the invention comprises a method of treating cancer in a patient, comprising administering to the patient a therapeutically effective amount of an extract of Scutellaria barbata D. Don and at least one additional agent, which antagonizes estrogen receptor activity.

In some embodiments, the invention comprises a method of treating cancer in a patient, comprising administering to the patient a therapeutically effective amount of an extract of Scutellaria barbata D. Don and at least one additional agent, which inhibits EGF receptor tyrosine kinase activity.

In some embodiments, the invention comprises a method of treating cancer in a patient, comprising administering to the patient a therapeutically effective amount of an extract of Scutellaria barbata D. Don and at least one additional agent, which inhibits VEGF receptor tyrosine kinase activity.

In some embodiments, the invention comprises a method of treating cancer in a patient, comprising administering to the patient a therapeutically effective amount of an extract of Scutellaria barbata D. Don and at least one additional agent, which inhibits RET tyrosine kinase activity.

In some embodiments, the invention comprises a method of treating cancer in a patient, comprising administering to the patient a therapeutically effective amount of an extract of Scutellaria barbata D. Don and at least one additional agent, which antagonizes endothelin A receptor activity.

In some embodiments, the invention comprises a method of treating cancer in a patient, comprising administering to the patient a therapeutically effective amount of an extract of Scutellaria barbata D. Don and at least one additional agent, which inhibits Src kinase or Abl kinase activity, or a combination thereof.

In some embodiments, the invention comprises a method of treating cancer in a patient, comprising administering to the patient a therapeutically effective amount of an extract of Scutellaria barbata D. Don and at least one additional agent, which inhibits CDK activity.

In some embodiments, the invention comprises a method of treating cancer in a patient, comprising administering to the patient a therapeutically effective amount of an extract of Scutellaria barbata D. Don and at least one additional agent, which inhibits MEK 1 or MEK 2 activity, or a combination thereof.

In some embodiments, the invention comprises a method of treating cancer in a patient, comprising administering to the patient a therapeutically effective amount of an extract of Scutellaria barbata D. Don and at least one additional agent, which inhibits aurora kinase activity.

Some embodiments described herein provide kit for the treatment of cancer comprising a pharmaceutically acceptable amount of a first chemotherapeutic agent comprising an extract of Scutellaria barbata D. Don and a second chemotherapeutic agent selected from the group consisting of an aromatase inhibitor, an androgen antagonizing agent, an agonist of gonadotropin releasing hormone, an estrogen receptor antagonist a tyrosine kinase inhibitor, an endothelin A receptor antagonist, an Src kinase inhibitor, an Abl kinase inhibitor, a CDK inhibitor, an inhibitor of MEK 1, MEK 2 or both, and an aurora kinase inhibitor. In some embodiments, the second chemothereapeutic agent is a tyrosine kinase inhibitor selected from an EGF receptor tyrosine kinase inhibitor, a VEGF receptor tyrosine kinase inhibitor, an RET tyrosine kinase inhibitor. In some embodiments, the second chemotherapeutic agent is selected from the group consisting of: (a) an aromatase inhibitor selected from anastrozole; (b) an androgen antagonist selected from bicalutamide; (c) an agonist of gonadotropin releasing hormone selected from goserelin; (d) an estrogen receptor antagonist selected from fulvestrant; (e) an EGF tyrosine kinase inhibitor selected from gefitinib or vandetanib; (f) a VEGF tyrosine kinase inhibitor selected from vandetanib and AZD2171; (g) an RET tyrosine kinase inhibitor selected from vandetanib; (h) an endothelin A receptor antagonist selected from ZD4054; (i) an inhibitor of Src kinase or Abl kinase selected from AZD0530; (j) a CDK inhibitor selected from AZD5438; (k) an inhibitor of MEK1 or MEK2 selected from AZD6244; and (1) an inhibitor of aurora kinase selected from AZD 1152. Some embodiments provide kit comprising a third chemotherapeutic agent. In some embodiments, the third chemotherapeutic agent is selected from the group consisting of: (a) an aromatase inhibitor selected from anastrozole; (b) an androgen antagonist selected from bicalutamide; (c) an agonist of gonadotropin releasing hormone selected from goserelin; (d) an estrogen receptor antagonist selected from fulvestrant; (e) an EGF tyrosine kinase inhibitor selected from gefitinib or vandetanib; (f) a VEGF tyrosine kinase inhibitor selected from vandetanib and AZD2171; (g) an RET tyrosine kinase inhibitor selected from vandetanib; (h) an endothelin A receptor antagonist selected from ZD4054; (i) an inhibitor of Src kinase or Abl kinase selected from AZD0530; (j) a CDK inhibitor selected from AZDS438; (k) an inhibitor of MEK1 or MEK2 selected from AZD6244; and (l) an inhibitor of aurora kinase selected from AZD1152.

Other uses and advantages of the present invention will be apparent to the person skilled in the art after having considered the description, including the drawings and claims, herein.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows dose-response curves showing the response of several solid cancer tumor cells to aqueous extract of the herb of this invention.

FIG. 2 shows dose-response curves showing the response of several breast solid cancer tumor cells to aqueous extract of the herb of the invention.

FIG. 3 shows dose-response curves comparing the response of breast solid cancer tumor cells and normal breast epithelium to aqueous extract of the herb of this invention.

FIG. 4 shows gel electrophoresis plate, which demonstrates that nuclear DNA disintegration occurs during apoptosis of solid tumor cancer cells in contact with aqueous extracts of the herb of this invention.

FIG. 5 shows the effect of the herb extract of the invention administered intraperitoneally (IP) on the tumors of mice in a xenograft model.

FIG. 6 shows the effect of the herb extract administered by oral gavages and in interaction with cyclophosphamide administered in low dose in the drinking water on the tumors of mice in a xenograft model.

FIG. 7 shows that the herb extract induces apoptosis without activating caspases.

FIG. 8 shows that the herb extract in cell cycle analysis arrests the cells at the G1 phase.

FIG. 9 shows that illustrates that BZL101 leads to oxidative DNA damage. Formation of 8-oxoguanine, the most ubiquitous marker of DNA oxidation, was quantified through flow cytometric analysis of fixed permeabilized cells incubated with avidin fluorescein, that was shown to bind relatively specifically to 8-oxoguanine. There is a clear increase in binding of avidin to BZL101 treated SKBr3 cells versus untreated cells.

FIG. 10 shows that the conversion of non-fluorescent CM-H₂DCFDA into fluorescent compound is indeed due to ROS. Incubation of cells with ROS scavenger N-acetyl-cysteine (NAC) prior to addition of BZL101 prevented most of the increase in ROS generation.

### DETAILED DESCRIPTION OF THE INVENTION

This invention relates to extract of Scutellaria barbata where the extract, when placed in contact with solid tumor cancer cells, inhibits the activity, that is the growth and/or proliferation, of the cells. The herb is selected from the species Scutellaria barbata D. Don of the Labiatae Family. Herba Scutellaria Barbata D. Don (Lamiaceae) of the Labiatae family—Ban Zhi Lian (BZL) is grown mainly in areas southeastern of the Yellow River (Huang Po) in the provinces of Sichuan, Jiangsu, Jiangxi, Fujian, Guangdong, Guangxi and Shaanxi but not exclusively. The plant is harvested in late summer and early autumn after it blooms (May-June). The aerial part is cut from the root. Only the aerial part (leaves and stems) is used for BZL101. The herb is dried in the sun and packed as a whole plant. The herb is received with no separation between leaves and stems.

As is described in the Detailed Description section, below, the herb is substantially more active in inhibiting the activity of different types of cancer cells. It is therefore a presently preferred aspect of this invention that the herbal extract obtained from the species Scutellaria barbata. It is a particularly presently preferred aspect of this invention that the herbal extract is obtained from Scutellaria barbata D. Don.

It is an aspect of this invention that the solid tumor cancer cell, the activity of which is inhibited by the herbal extract of this invention is a SKBR3 cell, a MCF7 cell, a MDA-MB231 cell, a BT474 cell or a MCNeuA cell (breast cancer cells), A549 cell, LLC cell (Lung Cancer cells), Panel cells, Panc02 cells (Pancreatic cancer cells), PC-3 cells LNCAP cells (Prostate Cancer cells), OVCAR cells, SKOV3 cells (Ovarian Cancer cells). In some embodiments of the invention, the cell line is in vivo, i.e. a xenograft of the tumor line is present in a mammalian model animal, such as a mouse, rat, dog, cat, sheep, goat or other mammal. Thus, the extract of Scutellaria barbata can be used as a standard for the evaluation of potential anti-cancer drugs.

In some embodiments, the invention comprises a method of treating cancer in a patient, comprising administering to the patient a therapeutically effective amount of an extract of Scutellaria barbata D. Don and at least one additional agent, which inhibits aromatase activity. In some embodiments, the extract of Scutellaria barbata and the additional agent are administered in the same dosage form. In some embodiments, the extract of Scutellaria barbata and the additional agent are administered sequentially or concurrently. In some embodiments, the agent that inhibits aromatase activity is anastrozole.

In some embodiments, the invention comprises a method of treating cancer in a patient, comprising administering to the patient a therapeutically effective amount of an extract of Scutellaria barbata D. Don and at least one additional agent, which antagonizes androgen activity. In some embodiments, the extract of Scutellaria barbata and the additional agent are administered in the same dosage form. In some embodiments, the extract of Scutellaria barbata and the additional agent are administered sequentially or concurrently. In some embodiments, the agent that antagonizes androgen activity is bicalutamide.

In some embodiments, the invention comprises a method of treating cancer in a patient, comprising administering to the patient a therapeutically effective amount of an extract of Scutellaria barbata D. Don and at least one additional agent, which agonizes gonadotropin releasing hormone activity. In some embodiments, the extract of Scutellaria barbata and the additional agent are administered in the same dosage form. In some embodiments, the extract of Scutellaria barbata and the additional agent are administered sequentially or concurrently. In some embodiments, the agent that agonizes gonadotropin releasing hormone activity is goserelin.

In some embodiments, the invention comprises a method of treating cancer in a patient, comprising administering to the patient a therapeutically effective amount of an extract of Scutellaria barbata D. Don and at least one additional agent, which antagonizes estrogen receptor activity. In some embodiments, the extract of Scutellaria barbata and the additional agent are administered in the same dosage form. In some embodiments, the extract of Scutellaria barbata and the additional agent are administered sequentially or concurrently. In some embodiments, the chemotherapeutic agent that antagonizes estrogen receptor activity is fulvestrant.

In some embodiments, the invention comprises a method of treating cancer in a patient, comprising administering to the patient a therapeutically effective amount of an extract of Scutellaria barbata D. Don and at least one additional agent, which inhibits EGF receptor tyrosine kinase activity. In some embodiments, the extract of Scutellaria barbata and the additional agent are administered in the same dosage form. In some embodiments, the extract of Scutellaria barbata and the additional agent are administered sequentially or concurrently. In some embodiments, the chemotherapeutic agent that inhibits EGF receptor tyrosine kinase activity is selected from the group consisting of: gefitinib or vandetanib.

In some embodiments, the invention comprises a method of treating cancer in a patient, comprising administering to the patient a therapeutically effective amount of an extract of Scutellaria barbata D. Don and at least one additional agent, which inhibits VEGF receptor tyrosine kinase activity. In some embodiments, the extract of Scutellaria barbata and the additional agent are administered in the same dosage form. In some embodiments, the extract of Scutellaria barbata and the additional agent are administered sequentially or concurrently. In some embodiments, the chemotherapeutic agent that inhibits VEGF receptor tyrosine kinase activity is selected from the group consisting of: vandetanib or AZD2171.

In some embodiments, the invention comprises a method of treating cancer in a patient, comprising administering to the patient a therapeutically effective amount of an extract of Scutellaria barbata D. Don and at least one additional agent, which inhibits RET tyrosine kinase activity. In some embodiments, the extract of Scutellaria barbata and the additional agent are administered in the same dosage form. In some embodiments, the extract of Scutellaria barbata and the additional agent are administered sequentially or concurrently. In some embodiments, the chemotherapeutic agent that inhibits RET tyrosine kinase activity is vandetanib.

In some embodiments, the invention comprises a method of treating cancer in a patient, comprising administering to the patient a therapeutically effective amount of an extract of Scutellaria barbata D. Don and at least one additional agent, which antagonizes endothelin A receptor activity. In some embodiments, the extract of Scutellaria barbata and the additional agent are administered in the same dosage form. In some embodiments, the extract of Scutellaria barbata and the additional agent are administered sequentially or concurrently. In some embodiments, the chemotherapeutic agent that antagonizes endothelin A receptor activity is ZD4054.

In some embodiments, the invention comprises a method of treating cancer in a patient, comprising administering to the patient a therapeutically effective amount of an extract of Scutellaria barbata D. Don and at least one additional agent, which inhibits Src kinase or Abl kinase activity, or a combination thereof. In some embodiments, the extract of Scutellaria barbata and the additional agent are administered in the same dosage form. In some embodiments, the extract of Scutellaria barbata and the additional agent are administered sequentially or concurrently. In some embodiments, the chemotherapeutic agent that inhibits Src kinase or Abl kinase activity, or a combination thereof, is AZD0530.

In some embodiments, the invention comprises a method of treating cancer in a patient, comprising administering to the patient a therapeutically effective amount of an extract of Scutellaria barbata D. Don and at least one additional agent, which inhibits CDK activity. In some embodiments, the extract of Scutellaria barbata and the additional agent are administered in the same dosage form. In some embodiments, the extract of Scutellaria barbata and the additional agent are administered sequentially or concurrently. In some embodiments, the chemotherapeutic agent that inhibits CDK activity is AZD5438.

In some embodiments, the invention comprises a method of treating cancer in a patient, comprising administering to the patient a therapeutically effective amount of an extract of Scutellaria barbata D. Don and at least one additional agent, which inhibits MEK 1 or MEK 2 activity, or a combination thereof. In some embodiments, the extract of Scutellaria barbata and the additional agent are administered in the same dosage form. In some embodiments, the extract of Scutellaria barbata and the additional agent are administered sequentially or concurrently. In some embodiments, the chemotherapeutic agent inhibits MEK 1 or MEK 2 activity, or a combination thereof, is AZD 6244.

In some embodiments, the invention comprises a method of treating cancer in a patient, comprising administering to the patient a therapeutically effective amount of an extract of Scutellaria barbata D. Don and at least one additional agent, which inhibits aurora kinase activity. In some embodiments, the extract of Scutellaria barbata and the additional agent are administered in the same dosage form. In some embodiments, the extract of Scutellaria barbata and the additional agent are administered sequentially or concurrently. In some embodiments, the chemotherapeutic agent that inhibits aurora kinase activity is AZD 1152.

The solid tumor cancer being treated is an epithelial cell cancer in another aspect of this invention.

The epithelial cell cancer is breast or ovarian cancer in a still aspect of this invention.

Some embodiments described herein provide kit for the treatment of cancer comprising a pharmaceutically acceptable amount of a first chemotherapeutic agent comprising an extract of Scutellaria barbata D. Don and a second chemotherapeutic agent selected from the group consisting of an aromatase inhibitor, an androgen antagonizing agent, an agonist of gonadotropin releasing hormone, an estrogen receptor antagonist, a tyrosine kinase inhibitor, an endothelin A receptor antagonist, an Src kinase inhibitor, an Abl kinase inhibitor, a CDK inhibitor, an inhibitor of MEK 1, MEK 2 or both, and an aurora kinase inhibitor. In some embodiments, the second chemothereapeutic agent is a tyrosine kinase inhibitor selected from an EGF receptor tyrosine kinase inhibitor, a VEGF receptor tyrosine kinase inhibitor, an RET tyrosine kinase inhibitor. In some embodiments, the second chemotherapeutic agent is selected from the group consisting of: (a) an aromatase inhibitor selected from anastrozole; (b) an androgen antagonist selected from bicalutamide; (c) an agonist of gonadotropin releasing hormone selected from goserelin; (d) an estrogen receptor antagonist selected from fulvestrant; (e) an EGF tyrosine kinase inhibitor selected from gefitinib or vandetanib; (f) a VEGF tyrosine kinase inhibitor selected from vandetanib and AZD2171; (g) an RET tyrosine kinase inhibitor selected from vandetanib; (h) an endothelin A receptor antagonist selected from ZD4054; (i) an inhibitor of Src kinase or Abl kinase selected from AZD0530; (j) a CDK inhibitor selected from AZD5438; (k) an inhibitor of MEK1 or MEK2 selected from AZD6244; and (l) an inhibitor of aurora kinase selected from AZD1152. Some embodiments provide kit comprising a third chemotherapeutic agent. In some embodiments, the third chemotherapeutic agent is selected from the group consisting of: (a) an aromatase inhibitor selected from anastrozole; (b) an androgen antagonist selected from bicalutamide; (c) an agonist of gonadotropin releasing hormone selected from goserelin; (d) an estrogen receptor antagonist selected from fulvestrant; (e) an EGF tyrosine kinase inhibitor selected from gefitinib or vandetanib; (f) a VEGF tyrosine kinase inhibitor selected from vandetanib and AZD2171; (g) an RET tyrosine kinase inhibitor selected from vandetanib; (h) an endothelin A receptor antagonist selected from ZD4054; (i) an inhibitor of Src kinase or Abl kinase selected from AZD0530; (j) a CDK inhibitor selected from AZD5438; (k) an inhibitor of MEK1 or MEK2 selected from AZD6244; and (l) an inhibitor of aurora kinase selected from AZD1152.

An aspect of this invention is a composition comprising a pharmaceutically acceptable carrier of excipient and an extract Scutellaria barbata.

The pharmaceutical composition comprises aqueous extracts of the above herb species in an aspect of this invention.

The pharmaceutical composition comprises alcohol extracts of the above species in a further aspect of this invention. In a presently preferred embodiment of this invention, the alcohol used to extract the herbs is ethyl alcohol.

The pharmaceutical composition comprises a combination of aqueous and alcohol extracts of the above species of herb in still another aspect of this invention.

Table 1 depicts the herb, from which extracts of this invention are obtained, listed by family, genus, species and tradition Chinese name, of this invention

TABLE 1

| Family | genus | Species | Chinese name | Herb part |
|---|---|---|---|---|
| Labiatae | Scutellaria | Barbata D. Don | Ban Zhi Lian | aerial |

Table 2A shows the degree of inhibition of the activity of several in vitro solid breast cancer tumor cell lines by the extract of this invention.

TABLE 2A

| MCF7 | SKBR3 | MDA-MB231 | BT474 | MCNeuA |
|---|---|---|---|---|
| ++ | ++ | ++ | + | ++ |

Table 2B shows the degree of inhibition of the activity of several in vitro solid cancer tumor cell lines by the extract of this invention.

TABLE 2B

| Lung | | Pancreatic | | Prostate | | Breast | | |
|---|---|---|---|---|---|---|---|---|
| Cancer | | Cancer | | Cancer | | Cancer | | Breast Normal |
| A549 | LLC | Panc1 | Panc02 | PC-3 | LNCaP | MCF7 | MCNeuA | HuMEC |
| + | ++ | + | ++ | + | + | ++ | ++ | − |
| 1424 | 492 | 1054 | 594 | 1035 | 1516 | 818 | 619 | |

− <50% inhibition,
+ 51-75% inhibition,
++ >75% inhibition,
IC₅₀ values (μg/ml)

The active ingredients in BZL101 are not known. The extract loses activity when reconstituted after drying, as well as when the extract is separated through physical and chemical means. The known chemical ingredients in the plant are scutellarin, scutelarein, carthamidin, isocarthamidin and wagonin.

### DEFINITIONS

As used herein, the term “method” refers to manners, means techniques and procedures for accomplishing a given task including, but not limited to, those manners, means techniques and procedures either known to, or readily developed from known manners, means, techniques and procedures by, practitioners of the chemical, pharmacological, biological biochemical, medical, and homeopathic arts.

As used herein, “inhibiting the activity” refers to slowing, preferably stopping, the growth and/or proliferation of cancerous cells, both in-place, i.e., growth and proliferation at the initial site of tumor formation, and proliferation by metastasis. Inhibiting the activity also encompasses, in fact it is the most preferred embodiment of this invention, killing cancerous cells.

As used herein, the term “cancer” refers to various types of malignant neoplasms, most of which can invade surrounding tissues, and may metastasize to different sites, as defined by Stedman's Medical Dictionary 25^{th} edition (Hensyl ed. 1990). Examples of cancers which may be treated by the present invention include, but are not limited to, brain, ovarian, colon, prostate, kidney, bladder, breast, lung, oral and skin cancers. In a presently preferred embodiment of this invention the cancer being treated is breast or ovarian cancer.

As used herein, the term “contacting” in the context of contacting a solid tumor cancer cell with an extract of this invention bringing an extract of this invention and a target cancer cell together in such a manner that the extract can affect the activity of the cell either directly or indirectly. As used herein, contacting refers to procedures conducted in vitro, i.e. cancerous cells which are the object of this invention are studied, outside a patient. Cells existing outside the patient can be maintained or grown in cell culture dishes. For cells outside the organism, multiple methods exist, and are well-known to those skilled in the art, to contact extract of this invention, with or without employment of various well-known transmembrane carrier techniques and direct cell microinjection

The term “in vivo” refers to contacting or treatment within a living organism, such as a living human or other mammal, such as a mouse or rat.

As used herein, an “extract” refers to the residue of soluble solids obtained after an herb, or selected part thereof is (1) for example, without limitation, chopped, crushed, pulverized, minced or otherwise treated to expose maximum surface area and (2) is placed in intimate contact with a liquid, usually, but not necessarily, under conditions of agitation and elevated temperature. Then, after a period of time under the foregoing conditions the mixture is filtered to remove solids and the liquid is removed by, for example but not limitation, evaporation or freeze drying. The liquid used to obtain an extract may be water or an organic solvent, for example, without limitation, an alcohol such as methyl, ethyl or isopropyl alcohol, a ketone such as acetone or methyl ethyl ketone (MEK), an ester such as ethyl acetate, an organochlorine compound such as methylene chloride, chloroform or carbon tetrachloride, a hydrocarbon such as pentane, hexane or benzene and the like. An extract may also be obtained by using a combination of these solvents with or without water.

As used herein, an “herb” refers to any plant that is reputed to have medicinal value in Traditional Chinese Medicine (TCM). That is, the use of extracts of various parts of these plants have been passed down from ancient to modern Chinese practitioners of herbal medicine as a means for treating various ailments. In some instances, clinical evidence using standard Western medical research protocols have verified the utility of some of the extracts. While each of the herbs, and parts thereof, that make up The pharmaceutical compositions of this invention have long been known in TCM, use of an extract or combination of extracts in a composition as disclosed herein for the treatment of solid tumor cancers, in particular breast and uterine cancer, has not been previously disclosed. In particular embodiments of the invention, the herb is Scutellaria barbata, especially Scutellaria barbata D. Don.

As used herein, the terms “treat”, “treating” and “treatment” refer to a method of alleviating or abrogating a solid tumor cancer and/or its attendant symptoms. In particular, the terms simply mean that the life expectancy of an individual affected with a cancer will be increased or that one or more symptoms of the disease will be reduced.

As used herein, “administer”, “administering” or “administration” refers to the delivery of an extract or extracts of this invention or of a pharmaceutical composition containing an extract or extracts of this invention to a patient in a manner suitable for the treatment of particular cancer being addressed.

As used herein, the term “mammal” refers to any mammal that is affected by a cancer, whether that cancer is autologous (ie. arises naturally in the mammal) or is of xenogenous (i.e. xenogenic) origin. The term “mammal” includes humans, as well as murine, canine, feline, equine, bovine, ovine, porcine and other mammalian species.

A “patient” refers to any higher organism that is susceptible to solid tumor cancers. Examples of such higher organisms include, without limitation, mice, rats, rabbits, dogs, cats, horses, cows, pigs, sheep, fish and reptiles. In particular examples, “patient” refers to a human being.

As used herein, the term “therapeutically effective amount” refers to that amount of an extract or combination of extracts of this invention which has the effect of (1) reducing the size of the tumor; (2) inhibiting (that is, slowing to some extent, preferably stopping) tumor metastasis; (3) inhibiting to some extent (that is slowing to some extent, preferably stopping) tumor growth; and/or, (4) relieving to some extent (or preferably eliminating) one or more symptoms associated with cancer (5) stabilizing the growth of the tumor, (6) extending the time to disease progression, (7) improving overall survival.

As used herein, a “pharmaceutical composition” refers to a mixture of one or more of the extracts described herein with other chemical components, such as physiologically acceptable carriers and excipients. The purpose of a pharmacological composition is to facilitate administration of an extract or extracts of this invention to patient.

As used herein, the term “pharmaceutically acceptable” means that the modified agent or excipient is generally regarded as acceptable for use in a pharmaceutical composition.

As used herein, a “physiologically acceptable carrier” refers to a carrier or diluent that does not cause significant irritation to an organism and does not abrogate the biological activity and properties of the administered composition.

As used herein, an “excipient” refers to an inert substance added to a pharmaceutical composition to further facilitate administration of an extract or extracts of this invention. Examples, without limitation, of excipients include calcium carbonate, calcium phosphate, various sugars and types of starch, cellulose derivatives, gelatin, vegetable oils and polyethylene glycols.

At one time, botanical agents were the most significant group of substances used by healers to treat patients. According to a WHO survey, 80% of the world's population still relies heavily on herbal medicine as their primary source of therapy. In Western culture one-quarter of the active components of currently prescribed drugs were first identified in plants and over half of the 50 most popular drugs today are derived from plant materials. In addition, over 60% of chemotherapeutic agents used in the treatment of cancer are derived from natural substances.

A useful strategy for the discovery of biologically active compounds from plants is the ethno-pharmacological approach which uses information about traditional medicinal uses of plants. The long history of a plant's use in treating a disorder, regardless of whether the disorder is well-characterized, e.g., skin rash, or is rather more nebulous, e.g., hot blood, is a clear indicator that something in the plant has some manner of beneficial effect on a disorder, otherwise the use of the plant would have faded in time. Furthermore, the fact that homeopathic practitioners have been administering the plant or an extract thereof to human patients for, often, centuries provides a compelling argument for the safety of the plant or its extracts in human beings.

Such alternative approaches to medicine are becoming more and more widely accepted and used in the United States as well to treat a broad spectrum of conditions as well as to maintain wellness. It is estimated that one in two Americans currently uses alternative therapies at one time or another. In particular, the most popular complementary or fully alternative approach to the treatment of their cancers by patients is botanical agents/herbal medicines.

Traditional Chinese medicine (TCM) is often the treatment modality of choice by cancer patients opting for an alternative approach to dealing with their ailment. Patients use TCM both as anti-cancer agents and to alleviate the side effects of standard chemotherapy. However, TCM lacks the scientifically sound methodology required of Western pharmacology and the use of TCM is often hit or miss in its effectiveness. There remains a need for the discovery of specific herbal extracts and combinations thereof that have a specific utility and for which there is scientific evidence as to why they work in that use. This invention provides such extract and compositions decoction.

### Pharmaceutical Compositions and Modes of Administrations

An extract of this invention can be administered to a patient either as a “tea,” without combination with any other substances or further manipulation, or it can be administered as a pharmaceutical composition where the extract is mixed with suitable carriers or recipient(s). In treating a patient exhibiting a disorder of interest, a therapeutically effective amount of the extract is administered. A therapeutically effective amount refers to that amount of the extract that results in amelioration of symptoms or a prolongation of survival in a patient, and may include destruction of a malignant tumor of a microbial infection.

When administered without combination with any other substances, the composition comprising extract of Scutellaria Barbata (especially Scutellaria Barbata D. Don) may be encased in a suitable capsule, such as a gelatin capsule. When administered in admixture with other excipients, adjuvants, binders, diluents, disintegrants, etc., the dry extract of Scutellaria Barbata may be compressed into a capsule or caplet in a conventional manner that is well-known in the art.

Toxicity and therapeutic efficacy of the extracts, i.e., determining the LD50 (the dose lethal to 50% of the population) and the EDSO (the dose therapeutically effective in 50% of the population) can be determined by standard pharmaceutical procedures in cell cultures or experimental animals. The dose ratio between toxic and therapeutic effects is the therapeutic index and it can be expressed as the ratio LD50/ED50. Extracts that exhibit large therapeutic indices are preferred. The data obtained from these cell culture assays and animal studies can be used in formulating a range of dosages for use in humans, in particular for internal use, that include ED50 with little or no toxicity. The dosage may vary within this range depending upon the dosage form employed and the route of administration utilized. In general, since the extracts used in the methods of this invention have been used in TCM, they are known to be relatively non-toxic to humans and therefore it is expected that they will exhibit large therapeutic indices.

For any extract used in the method of invention, the therapeutically effective dose can be estimated initially from cell culture assays. For example, a dose can be formulated in animal models to achieve a circulating plasma concentration range that includes the IC50 as determined in cell culture. Such information can be used to more accurately determine useful doses in humans. Levels in plasma may be measured, for example, by HPLC.

The exact formulation, route of administration and dosage can be chosen by the individual physician in view of the patient's condition and based on knowledge of TCM. (See e.g. Fingl et al., in THE PHARMACOLOGICAL BASIS OF THERAPEUTICS, 1975, Ch. 1, p. 1). It should be noted that the attending physician would know how and when to terminate, interrupt, or adjust administration due to toxicity, or organ dysfunction. Conversely, the attending physician would also know to adjust treatment to higher levels if the clinical response is not adequate. The severity of the condition may, for example, be evaluated, in part, by standard prognostic evaluation methods. Further, the dose and perhaps dose frequency will also vary according to the age, body weight, and response of the individual patient. A program comparable to that discussed above may be used in veterinary medicine.

If desired, standard western medicine techniques for formulation and administration may be used, such as those found in Remington's Pharmaceutical Sciences, 18^{th} ed., Mack Publishing Co., Easton, Pa. (1990). Suitable routes may include: oral, rectal, transdermal, vaginal, transmucosal, or intestinal administration; parenteral delivery, including intramuscular, subcutaneous, intramedullary injections; as well as intrathecal, direct intraventricular, intravenous, intraperitoneal, intranasal, or intraocular injections, to name a just a few. In particular embodiments, the extract of the invention is administered orally.

For injection, an extract of this invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate an extract herein use in the methods disclosed for the practice of this invention in dosages suitable for systemic administration is within the scope of the invention. With proper choice of carrier and suitable manufacturing practice, an extract of the present invention, in particular those formulated as solutions, may be administered parenterally, such as by intravenous injection. Likewise, an extract can be formulated, using pharmaceutically acceptable carriers well known in the art, into dosages suitable for oral administration. Such carriers enable extracts to be formulated as tablets, pills, capsules, liquids, gels, syrups, slurries, suspensions and the like, for oral ingestion by a patient to be treated.

Pharmaceutical compositions suitable for use in the present invention are compositions wherein an extract is contained in an effective amount to achieve its intended purpose. Determination of the effective amount is well within the capability of those skilled in the art, especially in light of the detailed disclosure provided herein. A pharmaceutical composition may contain suitable pharmaceutically acceptable carriers including excipients and auxiliaries that facilitate processing of the extracts into preparations that can be used pharmaceutically. The preparations formulated for oral administration may be in the form of tablets, dragees, capsules, or solutions. The pharmaceutical compositions of the present invention may be manufactured in a manner that is itself known, e.g., by means of convention mixing, dissolving, granulating, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutically formulations for parenteral administration include aqueous solutions of an extract in water-soluble form. Additionally, suspensions of an extract may be prepared as appropriate oily injection suspensions may contain substances that increase the viscosity of the suspension, such as sodium carboxymethyl cellulose, sorbitol, or dextran. Optionally, the suspension may also contain suitable stabilizers or agents that increase the solubility of an extract to allow for the preparation of highly concentrated solutions.

Pharmaceutical preparations for oral use can be obtained by combining an extract with solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum Arabic, talc, polyvinyl pyrrolidone, carpool gel, polyethylene glycol, and/or titanium dioxide, lacquer solutions, and suitable organic solvents or solvent mixtures. Dyestuffs or pigments may be added to the tablets or dragee coatings for identification or to characterize different combinations of extracts and/or doses.

Pharmaceutical preparations that can be used orally include push-fit capsules made of gelatin, as well as soft, sealed capsules made of gelatin and a plasticizer, such as glycerol or sorbitol. The push-fit capsules contain the extract in admixture with fillers such as lactose, binders such as starches, and/or lubricants such as talc or magnesium separate and, optionally, stabilizers. In soft capsules, the extract may be dissolved or suspended in suitable liquids, such as fatty oils, liquid paraffin, or liquid polyethylene glycols.

The dosage of BZL101 varies depending upon the tumor type, the stage of disease, the species of patient and the individual patient. In general, the amount of BZL101 administered to a human patient is equivalent to the soluble residue of about 0.1 g to about 1000 g of dried solid plant parts of BZL. In some embodiments, the effective dose is equivalent to about 1 to about 100 g of dried solid aerial plant parts of BZL, especially about 5 to about 50 g of dried solid aerial plant parts.

Chemotherapeutic Agents

In the context of the present invention, “chemotherapeutic agents” means a therapeutic compound that is used inhibit or combat the development, proliferation, or spread of neoplastic cells. By way of non-limiting example, a chemotherapeutic agent may include antineoplastic agents and endocrine therapies.

Antineoplastic agents may include alkylating agents; antimetabolites; plant alkaloids and other natural products such as vinca alkaloids, podophyllotoxins, colchicine derivatives, and taxanes; cytotoxic antibiotics such as actinomycines and anthracyclines; platinum compounds; methylhydrazines; and monoclonal antibodies.

Endocrine therapies may include hormones such as gonadotropin releasing hormone analogues; and hormone antagonists such as anti-estrogens, anti-androgens, and enzyme inhibitors.

Aromatase inhibitors

Aromatase is an enzyme which is thought to participate in the production of estrodiol, an estrogen, from adrenally-generated androstenedione, an androgen. It is has been shown that estrogen contributes to the development of certain cancers, such as breast cancer. Thus, one line of treatment has focused on lowering estrogen levels.

Estrogen may act by binding to estrogen receptors. Studies have shown that estrogen receptors are overexpressed in approximately 70% of breast cancer cases. Breast cancer cases where the estrogen receptor is overexpressed are deemed ER+.

The body of a pre-menopausal woman produces most of its estrogen in the ovaries. However, in post-menopausal women the production of estrogen in the ovaries is substantially reduced. The majority of estrogen in post-menopausal women is produced from adrenally-generated androgens converted by aromatase enzymes. It has been shown in vivo that inhibiting aromatase is an effective breast cancer treatment in post-menopausal women.

Two types of aromatase inhibitors have been classified. The first class is composed of steroidal aromatase inhibitors. These are thought to permanently bond with the aromatase enzyme complex. The result has been shown to be irreversible. The second class of aromatase inhibitors are classified as is non-steroidal aromatase inhibitors. They are thought to inhibit the action of the aromatase enzyme complex by competing with it. Their action is reversible.

### Anastrozole

Anastrozole is classified as a non-steroidal aromatase inhibitor. In vivo studies have shown that it significantly lowers serum estradiol concentrations.

It is orally administered as a tablet. Each tablet contains about 1 mg of the active pharmaceutical ingredient.

Anastrozole is indicated for the treatment of post-menopausal women with estrogen-receptor positive breast cancer. It is an adjuvant therapy for early breast cancer and a first-line treatment for post-menopausal women with locally advanced or metastatic breast cancers. It is also indicated for the treatment of post-menopausal women whose breast cancer has advanced following treatment with tamoxifen.

### Anti-Androgens

Androgens are hormones which participate in the development and maintenance of male sex characteristics. Testosterone is one example of an androgen. In vivo studies have demonstrated that prostatic carcinomas are sensitive to androgen levels and that these carcinomas respond favorably to treatments which counteract the effects of androgens or remove the sources of androgens.

Anti-androgens include hormone receptor antagonists. These compounds are thought to block the access of androgens to their appropriate receptors.

Bicalutamide

Bicalutamide is classified as a non-steroidal anti-androgen. It is thought that bicalutamide functions by binding to androgen receptors present in the cytosol. This prevents androgens from binding to these receptors.

It is orally administered as a tablet. Each tablet contains about 50 mg of the active pharmaceutical ingredient.

Bicalutamide is indicated for the treatment of stage D₂ metastatic prostate cancer in combination with a luteinizing hormone-releasing hormone analogue. It is also indicated as an adjuvant therapy for treatment of early stage prostate cancer.

### Anti-Estrogens

Estrogens are hormones which participate in the development and maintenance of female sex characteristics. Estradiol is one example of an estrogen. In vivo studies have demonstrated that breast, endometrial, and ovarian cancers are all sensitive to estrogen levels and that these cancers respond favorably to treatments which counteract the effects of estrogens or remove the sources of estrogens.

Estrogen may act by binding to estrogen receptors. Studies have shown that estrogen receptors are overexpressed in approximately 70% of breast cancer cases. Breast cancer cases where the estrogen receptor is overexpressed are deemed ER+.

Anti-estrogens include hormone receptor antagonists. These compounds are thought to block the access of estrogens to their appropriate receptors. It is hypothesized that the binding of estrogens to estrogen receptors may stimulate the proliferation of mammary cells. The resulting increase in cellular and DNA division leads to an increased chance of cancerous mutations.

Fulvestrant

Fulvestrant is an estrogen receptor antagonist. It has been shown that fulvestrant can bind to estrogen receptors with an affinity that is similar to that of estrodiol. This prevents estrogens from binding to these receptors. It has also been demonstrated that fulvestrant downregulates and degrades the estrogen receptor.

It is administered as an intramuscular injection. The patient receives 250 mg of the active pharmaceutical ingredient once a month.

Fulvestrant is indicated for use in postmenopausal women with metastatic estrogen receptor positive breast cancer whose cancers have advanced despite having already undergone an anti-estrogen therapy.

### Gonadotropin-Releasing Hormone Analogues

Gonadotropin-releasing hormone (GnRH) stimulates the synthesis of both luteinizing hormone (LH) and follicle-stimulating hormone (FSH). GnRH is secreted in pulses. In males the frequency of the pulses has been found to be constant. However, in females the frequency varies depending on the progression of the menstrual cycle.

Gonadotropin-releasing hormone agonists are analogues of GnHR. They differ from the natural hormone in that they have amino acid substitutions at positions 6 and 10. These compounds interact with the GnHR receptor, stimulating the release of LH or FSH. The intentional overstimulation of the GnHR receptor is called hypogonadalism. A prolonged period of hypogandalism results in the body downregulating the GnHR receptor. Following from the downregulation of the receptor is a decrease in the production of sex hormones. In vivo studies have shown that treatment with a GnHR agonist decreases the chance that the hormone sensitive cancer will recur.

### Goserelin

Goserelin is a gonadotropin-releasing hormone receptor analogue which agonizes the GnHR receptor. In vivo studies have demonstrated that treatment with goserelin causes an initial increase in production of FSH and LH and a corresponding increase in sex hormones. However, after about 14-21 days, the receptor is downregulated and LH or FSH levels fall, followed by a corresponding decrease in sex hormone levels.

It is administered via an implant which contains either 3.6 mg or 10.8 mg of the active pharmaceutical ingredient. The 3.6 mg implant is placed under the skin and releases a constant amount of the active pharmaceutical ingredient over a 28-week period. The 10.8 mg implant is placed under the skin and releases a constant amount of the active pharmaceutical ingredient over a 12-week period.

Both the 3.6 and 10.8 mg doses of goserelin are indicated for palliative treatment of patients with advanced prostate cancer. They are also indicated for use in patients with stage B2-C prostate cancer in combination with flutamide. The 3.6 mg dose is also indicated for palliative treatment of pre- and post-menopausal women with advanced breast cancer.

### Epidermal Growth Factor Receptor Tyrosine Kinase Inhibitors

A tyrosine kinase is an enzyme that can transfer a phosphate group from ATP to a tyrosine residue in a protein. On its cytoplasmic side epidermal growth factor receptor (EGFR) possesses a tyrosine kinase domain. Binding by its ligand stimulates EGFR to autophosphyoralate several tyrosine residues. This in turn causes the downstream activation of several other proteins associated with the EGFR. This cascade of activity can result in DNA synthesis and cell proliferation.

Mutations in the EGFR can result in overexpression of its autophosphorylation activity. This can lead to the overexpression of various downstream proteins. One such protein is the Ras protein. Ras is an anti-apoptosis protein. The overexpression of Ras is thought to lead to uncontrolled cell proliferation. Overexpression of EGFR's tyrosine kinase activity has been found in certain cancers, such as breast cancer and lung cancer.

Gefitinib

Gefitinib is hypothesized to be an inhibitor of the tyrosine kinase activity of EGFR. It is thought that gefitinib binds to EGFR's ATP-binding site thus inhibiting the tyrosine kinase domain from transferring a phosphate group from ATP to the receptor's tyrosine residues. This inhibition then leads to a decrease in Ras activity which increases cell apoptosis.

It is administered as a tablet. Each tablet contains 250 mg of the active pharmaceutical ingredient.

Gefitinib is indicated for use following the failure of platinum-based and docetaxel therapies in patients with locally advanced and metastatic non-small cell lung cancers (NSCLC).

Vandetanib

Vandetanib is also hypothesized to be an inhibitor of the tyrosine kinase activity of EGFR. It is currently undergoing clinical trials. Phase III trials are studying the effects of vandetanib on patients with NSCLC. Two trials are being conducted with a daily dose of 100 mg/day. A further two trials are being conducted using a daily dose of 300 mg/day.

### Vascular Endothelial Growth Factor Receptor Tyrosine Kinase Inhibitors

A tyrosine kinase is an enzyme that can transfer a phosphate group from ATP to a tyrosine residue in a protein. On its cytoplasmic side vascular endothelial growth factor receptor (VEGFR) possesses a tyrosine kinase domain. Binding by its ligand stimulates VEGFR to autophosphyoralate several tyrosine residues. This autophosphorylation allows Src to bind to the receptor which instigates an intracellular signaling cascade. Studies indicate that VEGFR is involved in angiogenesis.

Mutations in the VEGFR can result in overexpression of its autophosphorylation activity. This can lead to the overexpression of VEGFR. Overexpression of VEGFR is hypothesized to cause an increase in angiogenesis by increasing endothelial cell proliferation and migration. Additionally, overexpression of VEGFR is hypothesized to cause an increase in cellular permeability. Conversely, it is thought that decreasing the activity of VEGFR should decrease angiogenesis and cellular permeability.

Vandetanib

Vandetanib is hypothesized to be an inhibitor of the tyrosine kinase activity of VEGFR. It is currently undergoing clinical trials. Phase III trials are studying the effects of vandetanib on patients with N SCLC. Two trials are being conducted with a daily dose of 100 mg/day. A further two trials are being conducted using a daily dose of 300 mg/day.

AZD2171

AZD 2171 is hypothesized to be an inhibitor of the tyrosine kinase activity of all three VEGFR. It is currently undergoing Phase I clinical trials. Doses from 0.75 mg/kg/day to 1.5 mg/kg/day in studies involving human tumor xenographs in mice have inhibited tumor angiogenesis.

### RET Tyrosine Kinase Inhibitors

A tyrosine kinase is an enzyme that can transfer a phosphate group from ATP to a tyrosine residue in a protein. The receptor for the glial cell-line derived neurotrophic factor (GDNF) family of extracellular signaling molecules or ligands (GLF) is RET. RET possesses a tyrosine kinase domain. Upon binding by the GLF, RET autophosphyoralate several tyrosine residues. This binding instigates an intracellular signaling cascade. Studies indicate that RET is involved in cellular proliferation.

Vandetanib

Vandetanib is hypothesized to be an inhibitor of the tyrosine kinase activity of VEGFR. It is currently undergoing clinical trials. Phase III trials are studying the effects of vandetanib on patients with NSCLC. Two trials are being conducted with a daily dose of 100 mg/day. A further two trials are being conducted using a daily dose of 300 mg/day.

### Endothelin Receptor Antagonists

There are two different endothelin receptors. The first, ET_{A}R is activated by the binding of its ligand ET-1. It is thought to be involved in, among other processes, cell proliferation, mitogenesis, angiogenesis, and inhibition of apoptosis. Overexpression of ET_{A}R has been linked to the development and spread of certain cancers. Conversely, ET_{B}R is thought to promote cell apoptosis and decreased levels have been found in certain tumors. Additionally, ET-1 also binds to ET_{B}R. It is thought that under-expression of ET_{B}R leads to increased levels of free ET-1 which then binds to and activates ET_{A}R.

Endothelin receptor antagonists block access by the ligands to the receptors. There are two types of endothelin receptor antagonists. Selective antagonists affect only one of the receptors. Dual antagonists will block both receptors.

ZD4054

ZD4054 is a selective endothelin receptor antagonist. In studies it has been demonstrated that ZD4054 only binds to ET_{A}R with no detectable effect on ET_{B}R. It is hypothesized that by antagonizing ET_{A}R, ZD4054 should promote cell apoptosis and decrease tumor angiogenesis, invasion, and metastasis.

Phase I and II studies have involved orally dosing patients with metastatic hormone resistant prostate cancer with either 15 mg/day or 10 mg/day of ZD4054.

### Src Kinase Inhibitors

Src is a tyrosine kinase. A tyrosine kinase is an enzyme that can transfer a phosphate group from ATP to a tyrosine residue in a protein. Mutations in this gene have been linked to the development of various cancers. Overexpression of Src kinase has been found in chronic myeloid leukemia cells.

AZD0530

AZD0530 is hypothesized to be an inhibitor of Src kinase. It is thought that inhibition of Src kinase should reduce tumor invasion. It is currently being studied in patients with locally advanced or metastatic pancreatic cancer that cannot be removed by surgery, in patients previously treated metastatic colon cancer or rectal cancer, in patients with prostate cancer that did not respond to hormone, in patient with recurrent or metastatic head and neck cancer, in patients with extensive stage small cell lung cancer, in patients with metastatic or locally advanced breast cancer that cannot be removed by surgery, and in patients with advanced solid tumors.

### Abl Kinase Inhibitors

Abl is a tyrosine kinase. A tyrosine kinase is an enzyme that can transfer a phosphate group from ATP to a tyrosine residue in a protein. Abl has been shown to be involved in cell differentiation, division, and adhesion. Mutations in this gene have been linked to the development of various cancers. Overexpression of Abl kinase has been found in chronic myeloid leukemia cells.

AZD0530

AZD0530 is hypothesized to be an inhibitor of Src kinase. It is thought that inhibition of Src kinase should reduce tumor invasion. It is currently being studied in patients with locally advanced or metastatic pancreatic cancer that cannot be removed by surgery, in patients previously treated metastatic colon cancer or rectal cancer, in patients with prostate cancer that did not respond to hormone, in patient with recurrent or metastatic head and neck cancer, in patients with extensive stage small cell lung cancer, in patients with metastatic or locally advanced breast cancer that cannot be removed by surgery, and in patients with advanced solid tumors.

### Cyclin Dependent Kinase Inhibitors

Cyclin dependent kinase (CDK) is a family of serine/threonine protein kinases. A serine/threonine kinase is a protein which phospharylates other proteins on a serine or threonine residue. CDKs are activated by the binding of cyclin. CDKs have been shown to be involved in cell cycle regulation, DNA transcription, and mRNA processing.

AZD5438

AZD5438 is a reversible CDK inhibitor. It is hypothesized that by inhibiting CDK, AZD5438 will cause a cell to enter the G2, S-1, or G1 phases of the cell cycle. AZD5438 is currently in Phase I clinical trials with patients with advanced solid cancers.

### MEK1 and MEK 2 Inhibitors

MEK1 and MEK2 are dual-specificity kinases that appear to phosphorylate the tyrosine and threonine residues on MAP/ERK kinase 1 and 2. This phosphorylation is though to be essential to the mitogenic growth factor signal transduction cascade. It is thought that a mutation in MEK1 and MEK2 can lead to tumor cell proliferation.

AZD6244

AZD6244 is a selective inhibitor of both MEK1 and MEK2. Inhibition of MEK1 and MEK2 is thought to result in inhibition of growth factor-mediated cell signaling and tumor cell proliferation. AZD6244 is currently in Phase I trials and is being studied as an antiproliferation agent.

### Aurora Kinase Inhibitors

Aurora kinases are a family of serine/threonine kinases. A serine/threonine kinase is a protein which phospharylates other proteins on a serine or threonine residue. Aurora kinases are thought to take part in cellular division by controlling chromatid segregation. Mutations in the genes encoding these proteins have been shown to lead to segregation defects which can lead to the growth of tumors. Mutations in these genes have been found in multiple cancers.

AZD1152

AZD1152 is a specific inhibitor of Aurora kinases. It has been demonstrated that inhibition of Aurora kinases leads to an inhibition of spindle aggregation during mitosis. AZD1152 is currently in Phase I trials.

### Treatment of Cancers

Extracts of Scutellaria barbata D. Don may be used to treat solid tumors. Such tumors may include so-called estrogen receptor negative (ER⁻) breast cancer, estrogen receptor positive (ER⁺) cancer, and other solid tumor cancers. As used herein, the terms “estrogen receptor negative breast cancer” and “estrogen receptor positive breast cancers,” have meanings commonly ascribed to them in the art. The person skilled in the art will recognize that the terms “positive” and “negative” are relative terms describing levels of expression in a cell. In general, saying that a cell is “negative” for expression of a particular cell product means that the level of expression detected, if any, falls below a predetermined threshold. That threshold may be a detection limit, a background noise level or some arbitrary cutoff known and understood by one of skill in the art. As extracts of Scutellaria barbata D. Don do not necessarily require presence of ERα or ERβ in order to induce apoptosis in solid cancer cells, it is considered that doses of Scutellaria barbata D. Don may be used to treat, inter alia, either ER⁺ or ER⁻ breast cancers as well as other solid tumors. The dose of Scutellaria barbata D. Don extract may vary, however it is considered that a dose comprising the dry soluble portion of a hot water or ethanolic extract of about 1 to about 20,000 g, especially about 50 to about 10,000 g of dry aerial portions of Scutellaria barbata D. Don, is a therapeutically effective dose. When used in combination with another chemotherapeutic agents, the dose may be lowered to take advantage of synergetic effects. C that extracts of Scutellaria barbata D. Don may be used to treat include sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

### Kits

Also provided herein are kits for treatment of cancer. In some embodiments, the kits comprise two or more active chemotherapeutic agents, at least one of which comprises an extract of Scutellaria barbata D. Don. In some embodiments, a first chemotherapeutic agent comprises an extract of Scutellaria barbata D. Don in an oral dosage form. In some embodiments, the second chemotherapeutic agent is in an oral or parenteral dosage form. Suitable parenteral dosage forms include intravenous or intraperitoneal injections. Kits can also contain instructions for administration of the extract of Scutellaria barbata D. Don and/or the second chemotherapeutic agent. In some embodiments, the kit will contain sufficient extract of Scutellaria barbata D. Don for administration over 1, 2, 3, 4 or more weeks. In some embodiments, the dosage of extract of Scutellaria barbata D. Don will be divided into daily or twice daily doses. The daily dose of extract of Scutellaria barbata D. Don may vary depending on the second chemotherapeutic agent, the disease to be treated, the condition of the patient, etc. In general, the daily dose of extract of Scutellaria barbata D. Don will be the dried soluble extract of about 1 to 20,000 g, 10 to 10,000 g or 50 to 5000 g of dried aerial portion of Scutellaria barbata D. Don. The daily dose may be divided into 2, 3, 4 or more doses per day. When administered as a tea, the doses may be combined with a flavor or flavor-masking agent in order to enhance palatability.

Some embodiments described herein provide kit for the treatment of cancer comprising a pharmaceutically acceptable amount of a first chemotherapeutic agent comprising an extract of Scutellaria barbata D. Don and a second chemotherapeutic agent selected from the group consisting of an aromatase inhibitor, an androgen antagonizing agent, an agonist of gonadotropin releasing hormone, an estrogen receptor antagonist, a tyrosine kinase inhibitor, an endothelin A receptor antagonist, an Src kinase inhibitor, an Abl kinase inhibitor, a CDK inhibitor, an inhibitor of MEK 1, MEK 2 or both, and an aurora kinase inhibitor. In some embodiments, the second chemothereapeutic agent is a tyrosine kinase inhibitor selected from an EGF receptor tyrosine kinase inhibitor, a VEGF receptor tyrosine kinase inhibitor, an RET tyrosine kinase inhibitor. In some embodiments, the second chemotherapeutic agent is selected from the group consisting of: (a) an aromatase inhibitor selected from anastrozole; (b) an androgen antagonist selected from bicalutamide; (c) an agonist of gonadotropin releasing hormone selected from goserelin; (d) an estrogen receptor antagonist selected from fulvestrant; (e) an EGF tyrosine kinase inhibitor selected from gefitinib or vandetanib; (f) a VEGF tyrosine kinase inhibitor selected from vandetanib and AZD2171; (g) an RET tyrosine kinase inhibitor selected from vandetanib; (h) an endothelin A receptor antagonist selected from ZD4054; (i) an inhibitor of Src kinase or Abl kinase selected from AZD0530; (j) a CDK inhibitor selected from AZD5438; (k) an inhibitor of MEK1 or MEK2 selected from AZD6244; and (1) an inhibitor of aurora kinase selected from AZD1152. Some embodiments provide kit comprising a third chemotherapeutic agent. In some embodiments, the third chemotherapeutic agent is selected from the group consisting of: (a) an aromatase inhibitor selected from anastrozole; (b) an androgen antagonist selected from bicalutamide; (c) an agonist of gonadotropin releasing hormone selected from goserelin; (d) an estrogen receptor antagonist selected from fulvestrant; (e) an EGF tyrosine kinase inhibitor selected from gefitinib or vandetanib; (f) a VEGF tyrosine kinase inhibitor selected from vandetanib and AZD2171; (g) an RET tyrosine kinase inhibitor selected from vandetanib; (h) an endothelin A receptor antagonist selected from ZD4054; (i) an inhibitor of Src kinase or Abl kinase selected from AZD0530; (j) a CDK inhibitor selected from AZD5438; (k) an inhibitor of MEK1 or MEK2 selected from AZD6244; and (l) an inhibitor of aurora kinase selected from AZD1152.

### EXAMPLES

The herb from which the extracts of this invention were obtained were purchased from Shen Nong Herbs, Berkeley, Calif. Their identity was confirmed by reference to traditional pharmaceutical literature.

### Preparative Example 1

### Preparation of BZL101 for In Vitro and Mouse Experiments

Herbal extract was prepared as “boiled teas”, which is how most are prepared for use in traditional treatment regimes. Aqueous extracts were prepared by adding 7.5 g of dry ground herb to 125 ml distilled water, bringing the mixture to a boil and then simmering for 45 minutes. The mixture was cooled, during which period most of the solids sank to the bottom of the vessel. The aqueous layer was carefully decanted off of the residual solids, centrifuged for 5 minutes at 1500 rpm, sterile filtered through a 0.45 μm filter and stored at 4° C. until used. Generally, the extracts were tested within 1-2 weeks of preparation although most of the active extracts were found to retain activity after storage at 4° C. for several additional weeks. An aliquot of each extract was dried under vacuum and the dry weight of the water soluble substances extracted from each herb determined.

### Preparative Example 2

### Preparation of BZL101 for Human In Vivo Experiments

BZL101 is an aqueous extract of the aerial part of Scutellaria Barbata D. Don of the Lamiaceae family. Herba Scutellaria Barbata D. Don (Chinese pin yin transliteration—Ban Zhi Lian (BZL)) is grown mainly in areas southeastern of the Yellow River (Huang Po) in the provinces of Sichuan, Jiangsu, Jiangxi, Fujian, Guangdong, Guangxi and Shaanxi. The plant is harvested in late summer and early autumn after it blooms. The aerial part (leaves and stems) is cut from the root and is used as starting material (BZL). The aerial part of the herb is dried in the sun, packed as a whole plant. The herb is identified and verified through botanical, morphological and chemical characteristics to ensure purity.
A single dose of BZL101 is made through the following procedure and is termed BZL101 (Bionovo, Inc., Emeryville, Calif.).
   180 grams of the raw herb is ground to fine powder (25 mesh)
   The powder is mixed with 1800 ml of distilled water to form a slurry
   The slurry is than simmered at 70-72° C. for 60 minutes
   The extract is decanted and filtered through 22 μm filter
   The supernatant weight after extraction is 168 gm
   The volume of the solution is 1750 ml
   The extract is concentrated with a vacuum evaporator to reduce the volume of water to 350ml which constitutes a 5:1 concentration of the original solution
   The dry weight of soluble material in the extract is 12 gm
   It is packaged in a sterile, vacuum sealed container
   Testing for bacteria, yeast and heavy metals are preformed by an accredited laboratory

### Comparative Example 1

### In Vitro Inhibition of Cancer Cell Activity Cell Lines and Culture

The extract obtained in Preparative Example 1, above, was tested against four human breast cancer cell lines, SKBR3, MFC-7, MDA-MB23 1 and BT474, and one murine breast cancer cell line, MCNeuA. All lines were maintained in 90% DME supplement with 2.0 mom L-glutamine, 100 IU/ml penicillin, 100 μg/ml streptomycin and 10% heat-inactivated fetal bovine serum. Cells at 70-80% confluence were used for plating for growth inhibition assays.

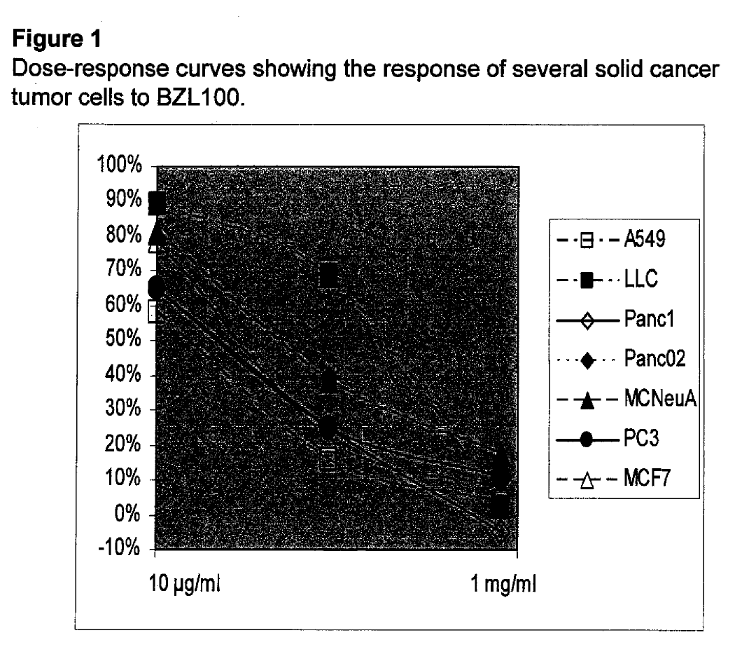

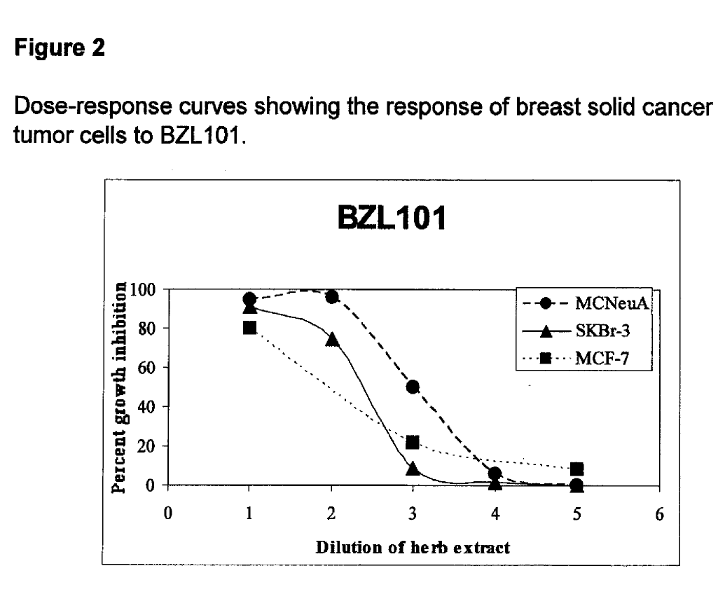

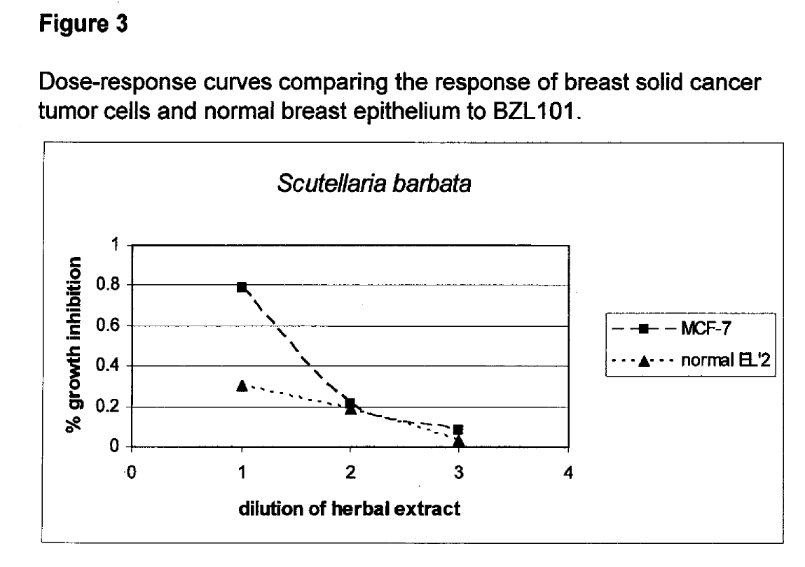

Cells were plated in 96-well flat bottom plates at 5,000 to 10,000 cells/well. The difference in number of cells plated adjusts for differences in the growth rates of these cell lines. Cells were allowed to adhere to the well walls overnight; then the extracts were added to triplicate wells at a 1:10 final dilution in culture medium for initial screening. For generating dose-response curves, serial 3-fold dilutions, starting at 1:10 dilution over 6 rows of wells were used. Water was added to the control wells at 1:10 dilution in culture medium. The plates were incubated at 37° C., 5% CO₂, for 3 days and then assayed for growth inhibition using a crystal violet assay (Bernhardt, G., et al., Standardized Kinetic Microassay to Quantify Differential Chemosensitivity on the Basis of Proliferative Activity, 1992, J. Cancer Res. Clin. Oncol., 118:35-43). Cells remaining adherent to the well walls were rinsed with PBS, the fixed cells were stained with 0.02% aqueous crystal violet (50 μl/well) for 30 minutes after which the wells were washed thoroughly with distilled water. The crystal violet stain bound by the cells was solubilized in 79% ethanol (100 μl/well) and the plates analyzed on a microplate reader (Molecular Devices) ay 595 nm. The percent inhibition was calculated as the average optical density of the control wells minus average optical density extract well divided by the average optical density of the control wells. Dose-response curves on SKBR3, MCF7 and MCNeuA cells for several of the extracts are shown in FIGS. 1-3. As can be seen, the concentration at which the extracts inhibited the activity of the cells by 50% (the IC50) ranged from over 1 mg/ml down to about 10 μg/ml.

Induction of Apoptosis

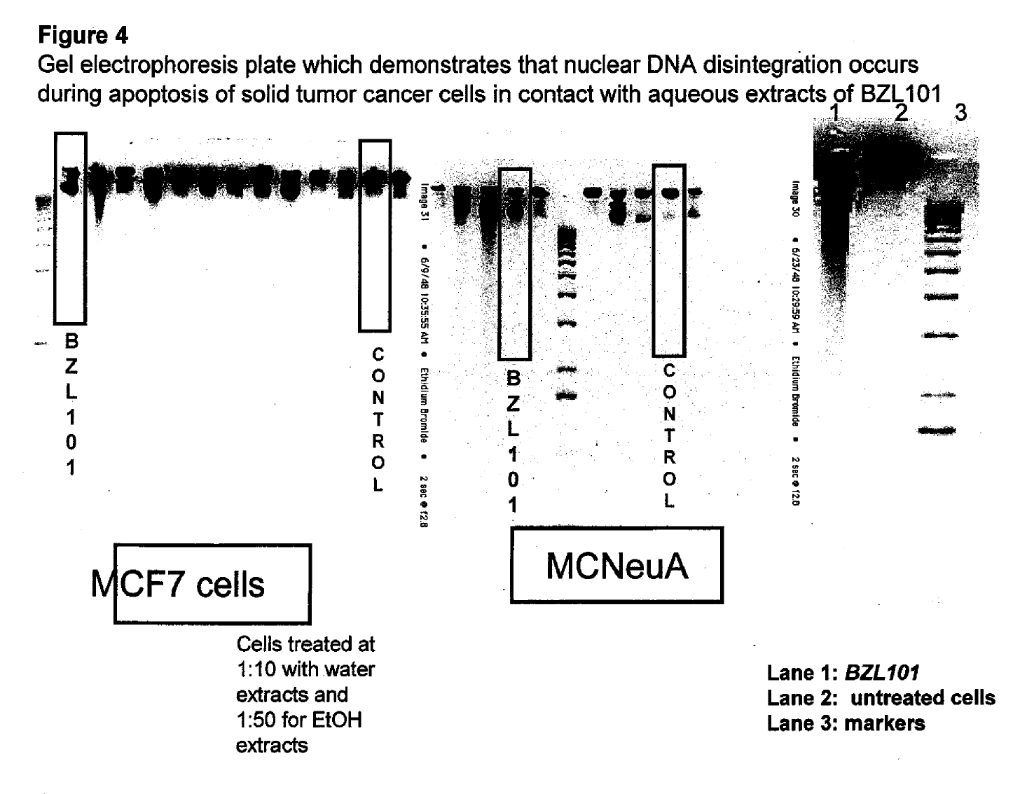

To assay for DNA fragmentation as a marker of apoptosis, a procedure for the isolation of genomic DNA that allows for the analysis of both high and low molecular weight DNA fragmentation during apoptosis was used. MCNeuA cells were plated at 5×10⁵ cells/well in 6-plates and allowed to adhere overnight. Aqueous herbal extracts were added to each well at a 1:10 and a 1:50 dilution. Sterile water, diluted 1:10 in culture medium, was added to the control wells. After 24 hours, the cells were visually examined under a microscope and morphological changes noted. Attached and floating cells were harvested, washed with cold PBS and embedded in lysis buffer (50 MM NaCl, 20 mM Tris HCl, pH 8.0, 20 mM EDTA, 0.5% sodium sarkosyl, 50 μg/ml Rnase A and 100 μg/ml proteinase K) for 1 hour at 37° C. The cells were then washed with PBS and distilled water and placed in the wells of a conventional 1% agarose gel and electrophoresed overnight at approximately 1 V/cm. The gels were then stained with ethidium bromide and photographed under w transillumination to give intense images. The images obtained are shown in FIG. 4.

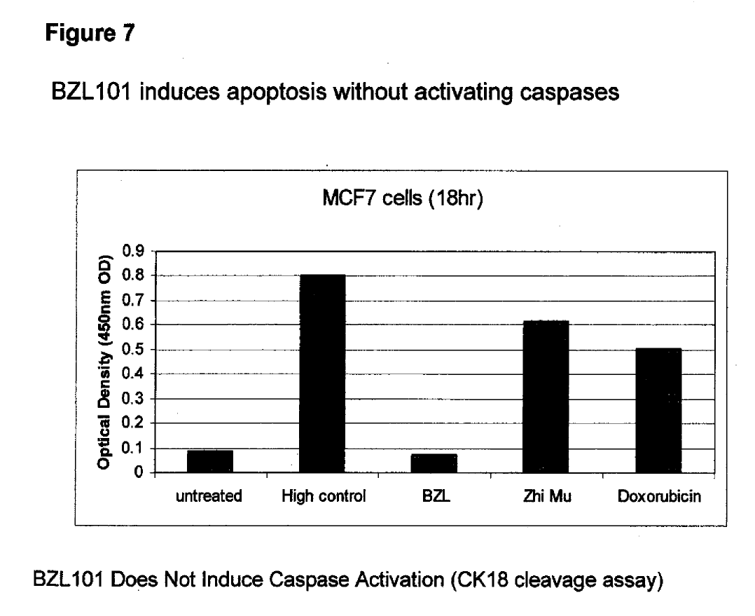

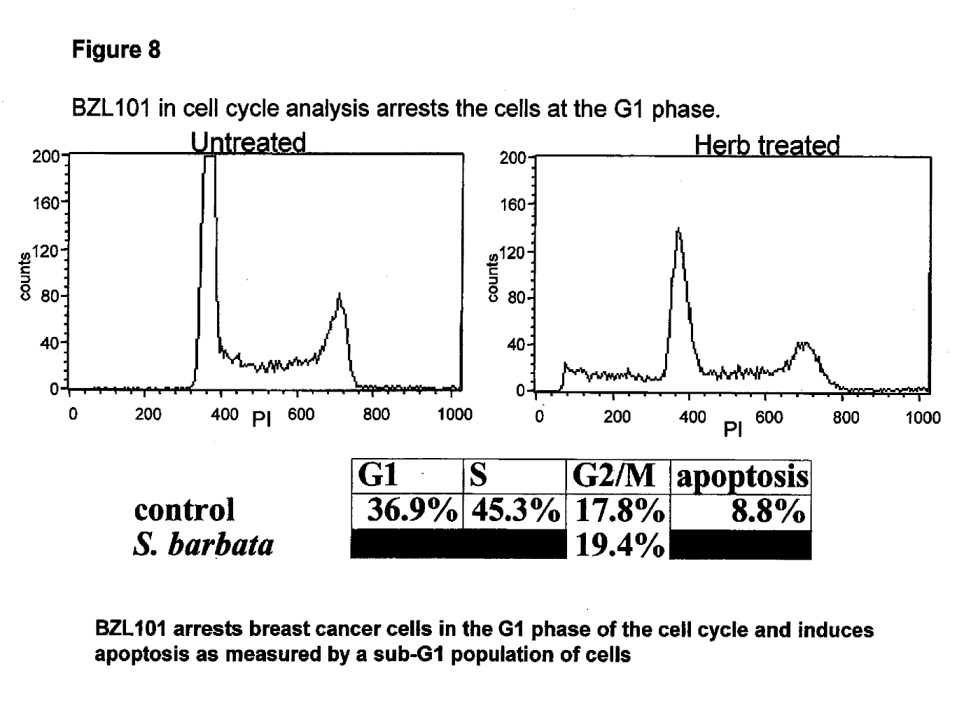

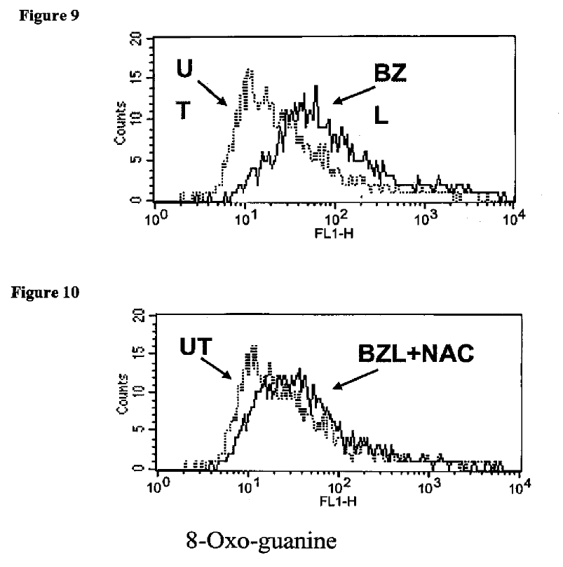

BZL101 was evaluated for antiproliferative activity on five breast cancer cell lines (SK-BR-3, MCF7, MDA-MB-231, BT474, and MCNeuA). These cell lines represent important prognostic phenotypes of breast cancer expressing a range of estrogen and HER2 receptors. BZL101, tested at a 1:10 dilution (15 μg/ml), demonstrated >50% growth inhibition on four of the five cell lines (Campbell, 2002). BZL101 showed >50% growth inhibition on a panel of lung, prostate and pancreatic cancer cell lines. BZL101 at the same dose did not cause >25% of growth inhibition on normal human mammary cells (HuMEC), demonstrating selectivity to cancer cells (Table 3). Moreso, BZL101 had a mild mitogenic effect on normal human lymphocytes. In cell cycle analysis, BZL101 caused an S phase burst and G1 arrest. (See FIG. 8). BZL101 also attenuated mitochondrial membrane potential causing caspase-independent high molecular grade (HMG) apoptosis. (See FIG. 7).

The results of this in vitro experiment are summarized in Table 3, below.

TABLE 3

| | | | | | | Breast | | | | | |
|---|---|---|---|---|---|---|---|---|---|---|---|
| | | | | | | | | | MDA- | | |
| Lung | | Pancreas | | Prostate | | | | | MB- | | |
| A549 | LLC | Panc-1 | Panc 02 | PC-3 | LNCaP | MCF7 | BT474 | SKBR3 | 231 | MCNeuA | HuMEC |
| + | + | + | ++ | + | + | ++ | + | ++ | + | ++ | − |

Table 3: In vitro growth inhibitory effect of BZL101 aqueous extract of Scutellaria Barbata 1:10 dilution − <50% inhibition, + 51-75% inhibition, ++ >75% inhibition. BZL is active on all cancer cell lines but is not active on HuMECs.

### Example 1

### In Vivo (IP) Efficacy of BZL101 in a Mouse Xenograft Model

In order to demonstrate the efficacy of BZL101 in the in vivo treatment of cancer, BZL101 was evaluated in a mouse xenograft model.

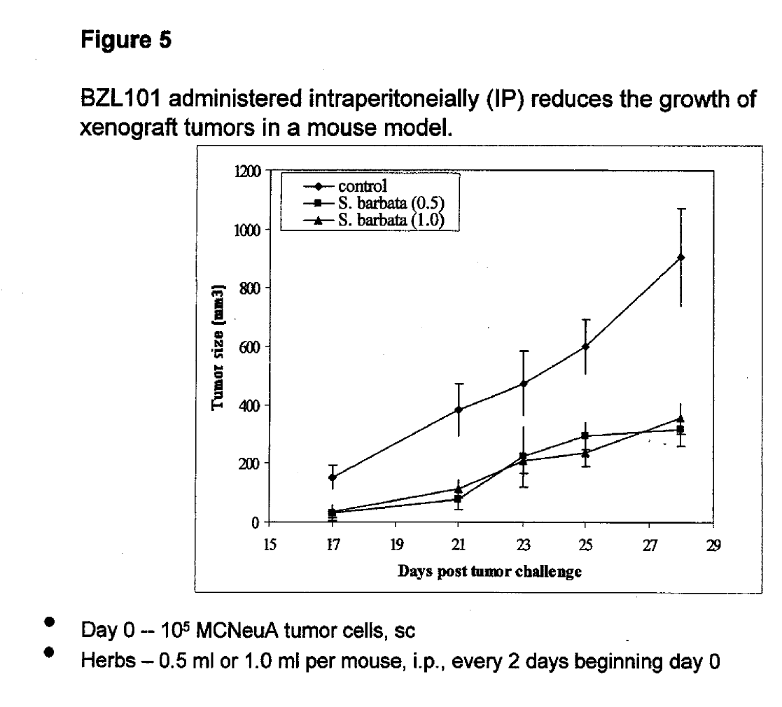

BZL101 was active via intraperitoneal (IP) administration in preventing tumor formation in a mouse xenograft model (FIG. 5). BZL101 was prepared as described in Preparative Example 1, above. Cells (10⁵) of MCNeuA cells were injected subcutaneously into mice on day 0. BZL101 (0.5 ml or 1.0 ml) or control was administered to each mouse IP every two days. Tumor size (mm³) was estimated on the 17^{th}, 21^{st}, 23^{rd}, 25^{th}, and 28^{th} day post administration. The results of this study, show in FIG. 5, demonstrate that BZL101 inhibited xenograft, suggesting that BZL101 can be an effective treatment for solid tumors in vivo.

### Example 2

### In Vivo (Oral) Efficacy of BZL101 in a Mouse Xenograft Model

In order to further evaluate the effect of the herb extract in vivo, BZL101 alone, BZL101 plus cyclophosphamide and cyclophosphamide alone were orally administered to mice having subcutaneous cancer xenografts.

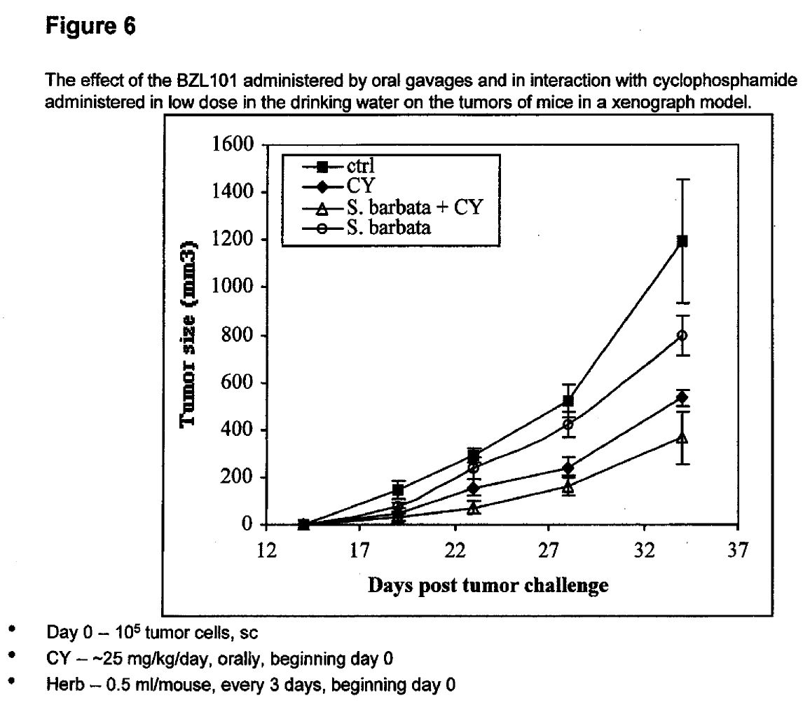

As in Example 1, above, 10⁵ cells were administered to each animal subcutaneously on Day 0. The animals were divided into four groups. The control group received only normal drinking water. The cyclophosphamide only group received 25 mg/Kg/day of cyclophosphamide in their drinking water. The BZL101 only group received 0.5 ml of BZL101 by oral gavage on Day 0 and every third day after that. The combination group received 0.5 ml/day BZL101 by oral gavage on Day zero and every third day after that, as well as 25 mg/Kg/day of cyclophosphamide in their drinking water. The results of this experiment are shown in FIG. 6.

From the results in FIG. 6, it can be seen that, as expected, cyclophosphamide alone inhibited tumor growth as compared to the control. BZL101 alone also demonstrated tumor growth inhibition. And the combination of BZL101 and cyclophosphamide inhibited tumor growth to a greater extent than did either BZL101 or cyclophosphamide alone. These results demonstrate in vivo efficacy of BZL101 in the treatment of solid tumors and suggest that BZL101 is probably effective in the treatment of solid tumors in general.

### Example 3

### Efficacy of BZL101 in Humans

In order to demonstrate the safety and clinical activity of oral BZL101, an aqueous extract from Scutellaria Barbata D. Don was studied in human patients with advanced breast cancer.

Eligible patients had histologically confirmed metastatic breast cancer and measurable disease. Patients did not receive any other chemotherapy, hormone therapy or herbal medicine during the trial. Patients received 350 ml (equivalent to 12 grams dry solubles BZL) BZL101 extract per day until disease progression, toxicity or personal preference caused them to discontinue. The primary endpoints were safety, toxicity and tumor response.

Twenty-one patients were enrolled and received BZL101. Mean age was 54 years (30-77) and mean number of prior treatments was 3.9 (0-10). There were no hematologic, nor grade III or IV non-hematologic, adverse events (AEs). Some patients reported grade I and II adverse events, such as nausea, diarrhea, headache, flatulence, vomiting, constipation, and fatigue. Sixteen patients were evaluable for response. Four of the 16 patients had stable disease (SD) for >90 days (25%) and 3/16 had SD for >180 days (19%). Five patients had minor objective tumor regression, one of which was I mm short of a PR based on RECIST criteria.

Patients were enrolled at the University of California, San Francisco Carol Franc Buck Breast Care Center and the Cancer Research Network in Plantation, Fla. between August 2001 and November 2004 and signed an informed consent approved by local institutional review boards. All patients were ≧18 years old with histologically confirmed diagnosis of breast cancer and clinical evidence of metastatic involvement. Patients with solitary metastases required biopsy confirmation of metastatic disease. All patients had completed prior therapies and had adequate time to recover sufficiently from the toxicities associated with prior anticancer treatments. A life expectancy of 6 months and Karnofsky performance status of 80% or better was required. Nutritional or up to five times recommended daily allowance (RDA) vitamin supplementation were permitted; but concomitant use of non-study herbal agents was prohibited. Patients were excluded from the study for the following: extensive liver involvement (>50% of liver parenchyma), lymphangitic pulmonary involvement, central nervous system involvement or spinal cord compression not stabilized by therapy for >3 months, a history of multiple or severe food or medicine allergies and organ or marrow dysfunction as defined by creatinine >2.0 mg/dl, total bilirubin >1.7 mg/dl, white blood cell count <2,500 cells/μL and platelet count <75,000 mm³.

Safety monitoring was done on a continuous basis and patients were seen by a physician for examination at baseline at every Y weeks. Adverse events were graded using Common Toxicity Criteria version 2, assigned a category by organ system and coded in relation to study drug as remote, possible, probably or definitely related. Baseline tumor assessments were done within 14 days of initiation of study drug and every three months. Responses were assessed using RECIST criteria. Study drug was administered at every visit, and at this visit compliance and a review of dosages taken was performed. BZL101 extract was provided as a liquid in a sealed and labeled aluminum packet containing a full daily dose that was administered in a split dose twice a day. Daily BZL extract was administered until the determination of tumor progression or dose limiting toxicity was encountered, or until the subject decided to voluntarily discontinue, in which case, the reason for discontinuation was obtained.

### Results

Patient Characteristics

A total of 22 patients with advanced breast cancer consented to the study and 21 patients were treated with at least one dose of oral BZL101 and included in the safety analysis. The last patient accrued to the study was not treated with BZL101 as funding for the study from the California Breast Cancer Research Program had ended and the expiration date for the study medication was nearing. Sixteen of the patients were treated for 28 days or more and evaluable according to the Response Evaluation Criteria in Solid Tumors (RECIST). Nine subjects discontinued study medication due to patient preference, and twelve patients were removed from the study due to progression based on RECIST criteria. None of the patients were removed from the study due to either grade III or IV adverse events categorized according to the National Cancer Institute (NCI) Common Toxicity Criteria (CTC) version 2. See Table 4 for a summary of study participants and Table 5 for a summary of selected patient characteristics.

TABLE 4
Summary of Study Participants

| | | | |
|---|---|---|---|
| (1) | Study Participants Consented | (2) | 22 |
| (3) | Consented but not Treated with BZL101 | (4) | 1* |
| (5) | Included in Safety Analysis | (6) | 21 |
| (7) | Evaluable by RECIST Criteria | (8) | 16 |
| (9) | Off Study Due to Patient Preference | (10) | 9 |
| (11) | Off Study Due to Progression of Disease | (12) | 12 |
| (13) | Off Study Due to Grade III or IV Toxicity | (14) | 0 |

*Inventory of study medication was nearing expiration and funding for the study had ended.

TABLE 5
Summary of Baseline Characteristics: Age, Height, Weight,
Race or Ethnicity

| | | |
|---|---|---|
| Age | | |
| Mean | 54.3 | years |
| Median | 55.5 | years |
| Range | 30-77 | years |
| Height | | |
| Mean | 65.2 | inches |
| Median | 65.0 | inches |
| Range | 62-68 | inches |
| Weight | | |
| Mean | 137.1 | pounds |
| Median | 139 | pounds |
| Range | 108-165 | pounds |
| Race or Ethnicity | | |

| | |
|---|---|
| Caucasian | 13 (59%) |
| African American | 2 participants (9%) |
| Hispanic | 1 participant (5%) |
| Asian | 1 participant (5%) |
| Native American | 1 participant (5%) |
| Unknown | 4 participants (18%) |

Safety Data

There were no deaths, serious adverse events or hematological adverse effects attributed to the study medication BZL101. There were no grade III or IV toxicities that were classified as possibly, probably or definitely related to BZL101.

Efficacy

Of the 21 patients who were treated with study medication, 16 patients were on the trial for 28 days or more and evaluable for response. Four of the 16 patients (25%) had stable disease for >90 days and 3/16 (19%) had stable disease for >180 days. Five patients had some degree of objective tumor regression, classified as a minimal response (<10% but <30 reduction in diameter sums). One of these responses was 1 mm short of a partial remission based on RECIST criteria. The average number of prior therapies for metastatic disease prior to treatment with the study medication, for patients who took at least one dose of BZL101, was 3.9 (See Table 6).

TABLE 6
Response to Treatment Based on RECIST Criteria

| | | | | | | Recist Criteria (Months) | | | | | |
|---|---|---|---|---|---|---|---|---|---|---|---|
| | | | | | | NE = Not evaluable | | | | | |
| | | | | | Prior | PD = Progressive Disease, | | | | | |
| | | | | | Therapies | SD = Stable Disease, | | | | | |
| | | | | | After | PR = Partial Remission, | | | | | |
| | | | | | Diagnosis of | CR = Complete Remission | | | | | |
| | | | Days | | Metastasis | MR = Minimal Response, | | | | | |
| | | | on | Reason for | But Before | >0% and <30% reduction | | | | | |
| Patient # | Age | On Study | Study | Discontinuation | BZL101 | NE | PD | SD | PR | CR | MR |
| 2001 | 48 | Aug. 28, 2001-Mar. | 184 | Progression | CMF | | 6 | 3 | | | |
| | | 14, 2002 | | | Capecitabine | | | | | | |
| 2002 | 30 | Oct. 02, 2001-Oct. | 25 | Progression | Goserelin | | <1 | | | | |
| | | 26, 2001 | | | Anastrozole | | | | | | |
| | | | | | Tamoxifen | | | | | | |
| | | | | | Targretin | | | | | | |
| | | | | | trial | | | | | | |
| | | | | | Docetaxel | | | | | | |
| | | | | | AC | | | | | | |
| | | | | | High dose | | | | | | |
| | | | | | chemo | | | | | | |
| | | | | | Capecitabine | | | | | | |
| | | | | | VEGF Trial | | | | | | |
| | | | | | Exemestane | | | | | | |
| 2003 | 50 | Oct. 30, 2001-Apr. | 151 | Pt | Anastrozole | | | 5 | | | 2, 3, 4 |
| | | 17, 2002 | | Preference | Tamoxifen | | | | | | |
| 2004 | 77 | Dec. 20, 2001-Sep. | 259 | Progression | None | | 9 | 6 | | | 3 |
| | | 05, 2002 | | | | | | | | | |
| 2005 | 64 | Mar. 07, 2002-Apr. | 36 | Pt | None | | | 1 | | | |
| | | 11, 2002 | | Preference | | | | | | | |
| 2006 | 59 | Oct. 31, 2002-Jan. | 71 | Pt | CAF | NE | | | | | |
| | | 09, 2003 | | preference | Tamoxifen | | | | | | |
| | | | | | CMF | | | | | | |
| | | | | | Paclitaxel | | | | | | |
| | | | | | Carboplatin + | | | | | | |
| | | | | | Etoposide | | | | | | |
| | | | | | Capecitabine | | | | | | |
| 2007 | 60 | Dec. 09, 2002-Dec. | 16 | Pt | Docetaxel | NE | | | | | |
| | | 25, 2002 | | Preference | Trastuzamab | | | | | | |
| | | | | | Cisplatin | | | | | | |
| | | | | | Capicitabine | | | | | | |
| | | | | | Liposomal | | | | | | |
| | | | | | doxirubicin | | | | | | |
| | | | | | Gemcitabine | | | | | | |
| 2008 | 52 | Jun. 24, 2003-Aug. | 59 | Pt | Exemestane | NE | | | | | |
| | | 21, 2003 | | Preference | Tamoxifen | | | | | | |
| | | | | | Capecitabine | | | | | | |
| 2009 | 34 | Sep. 12, 2003-Oct. | 41 | Progression | Doxorubicin | | 1.5 | | | | |
| | | 28, 2003 | | | Paciltaxel | | | | | | |
| | | | | | Docetaxel | | | | | | |
| 2010 | 56 | Jun. 26, 2003-Jun. | 1 | Pt | Tamoxifen | NE | | | | | |
| | | 27, 2003 | | Preference | CAF | | | | | | |
| | | | | | Traztuzamab | | | | | | |
| | | | | | Gemcitabine | | | | | | |
| | | | | | Letrozole | | | | | | |
| | | | | | Fulvestrant | | | | | | |
| 2011 | 48 | Apr. 21, 2004-Jul. | 93 | Progression | Docetaxil | | 3 | | | | |
| | | 23, 2004 | | | Gemcitabine | | | | | | |
| 2012 | | Nov. 08, 2004-Nov. | 6 | Pt | Letrozole | NE | | | | | |
| | | 15, 2004 | | Preference | Fulvestrant | | | | | | |
| | | | | | Carboplatin + | | | | | | |
| | | | | | Docetaxel | | | | | | |
| | | | | | Zoledronic | | | | | | |
| | | | | | acid | | | | | | |
| 3001 | 54 | Feb. 28, 2002-Apr. | 51 | Progression | Vinorelbine | | 1.5 | | | | |
| | | 19, 2002 | | | Traztuzamab | | | | | | |
| | | | | | Capecitabine | | | | | | |
| 3002 | 48 | Feb. 28, 2002-Mar. | 7 | Pt | Anastrazole | NE | | | | | |
| | | 07, 2002 | | Preference | Letrazole | | | | | | |
| 3003 | 59 | Mar. 01, 2002-Nov. | 260 | Progression | Liposomal | | 9 | | | | 1 |
| | | 15, 2002 | | | doxorubicin + | | | | | | |
| | | | | | Paclitaxel | | | | | | |
| 3004 | 59 | Mar. 04, 2002-Apr. | 33 | Progression | Tamoxifen | | | | | | 1 |
| | | 06, 2002 | | | Docetaxel | | | | | | |
| | | | | | Letrazole | | | | | | |
| 3005 | 60 | Mar. 29, 2002-May | 42 | Progression | Tamoxifen | | 1 | | | | |
| | | 12, 2002 | | | Letrozole | | | | | | |
| | | | | | Anastrozole | | | | | | |
| | | | | | Vinorelbine + | | | | | | |
| | | | | | Capecitabine | | | | | | |
| | | | | | NFL | | | | | | |
| 3006 | 56 | Apr. 17, 2002-Jul. | 63 | Progression | Tamoxifen | | 2 | | | | 1 |
| | | 01, 2002 | | | Liposomal | | | | | | |
| | | | | | doxorubicin | | | | | | |
| | | | | | NFL | | | | | | |
| | | | | | Anastrozole | | | | | | |
| | | | | | Trastuzamab | | | | | | |
| | | | | | Vinorelbine | | | | | | |
| | | | | | Gemcitabine | | | | | | |
| | | | | | Capecitabine | | | | | | |
| 3007 | 54 | Sep. 13, 2002-Nov. | 59 | Progression | TAC | | 2 | | | | |
| | | 11, 2002 | | | Tamoxifen | | | | | | |
| | | | | | Doxorubicin | | | | | | |
| | | | | | Trastuzamab | | | | | | |
| | | | | | Docetaxel | | | | | | |
| | | | | | CMF | | | | | | |
| | | | | | Vinorelbine | | | | | | |
| | | | | | Capecitabine | | | | | | |
| | | | | | Fulvestrant | | | | | | |
| 3008 | 67 | Apr. 09, 2004-May | 38 | Pt | Paclitaxel | | | 1 | | | |
| | | 17, 2004 | | Preference | Vinorelbine + | | | | | | |
| | | | | | Capecitabine | | | | | | |
| | | | | | Pfizer | | | | | | |
| | | | | | clinical trial | | | | | | |
| | | | | | Docetaxel | | | | | | |
| | | | | | Gemcitabine | | | | | | |
| | | | | | Liposomal | | | | | | |
| | | | | | doxorubicin | | | | | | |
| 3009 | 45 | May 24, 2004-Aug. | 95 | Progression | None | | 3 | | | | |
| | | 27, 2004 | | | | | | | | | |
| 3010 | 59 | Not | 0 | | Tamoxifen | NE | | | | | |
| | | treated | | | Anastrozole | | | | | | |
| | | | | | Capecitabine | | | | | | |
| | | | | | Vinorelbine | | | | | | |
| | | | | | Liposomal | | | | | | |
| | | | | | doxorubicin + | | | | | | |
| | | | | | Gemcitabine | | | | | | |
| | | | | | Carboplatin + | | | | | | |
| | | | | | Paclitaxel | | | | | | |
| | | | | | Fulvestrant | | | | | | |
| | | | | | Toremifene | | | | | | |
| | | | | | Letrozole | | | | | | |
| | | | | | Zoledronic | | | | | | |
| | | | | | Acid | | | | | | |

NFL mitoxantrone, 5-fluorouracil, leucovorin
CMF cyclophosphamide, methotrexate, fluorouracil
CAF cyclophosphamide, adriamycin, fluorouracil
TAC docetaxel, adriamycin (doxorubicin), cyclophosphamide
AC adriamycin (doxorubicin), cyclophosphamide

In a modified RECIST evaluation, where all measurable lesions were included as evaluable, one patient had a partial response or a reduction of 31% in the sum of the longest tumor diameter of all measurable lesions after 7 weeks of treatment and a reduction of 33% after 11 weeks of treatment (Table 7).

TABLE 7
Patient #2003 Response to Treatment Based on Modified RECIST Criteria

| | Lesion 1 | Lesion 2 | Lesion 3 | Lesion 4 | |
|---|---|---|---|---|---|
| | Site and | Site and | Site and | Site and | Total |
| | Method | Method | Method | Method | Measurable |
| DATE | Measurement | Measurement | Measurement | Measurement | Disease |
| #2003 | Site: Lymph | Site: Lymph | Site: Lymph | Site: | Total Baseline |
| Baseline | Node-Left | Node-Anterior | Node-Left | Vertebrae/Pelvis | Diameters = |
| Oct. 30, 2001 | Subclavian | Cervical | Subclavian, Post | Method: Pelvic | 5.8 cm |
| | Method: | Method: | Cervical | CT scan | |
| | Palpation | Palpation | Method: | Bony metastases | |
| | Measurement: | Measurement: | Palpation | | |
| | 3.0 × 2.5 cm | 2.0 × 2.0 cm | Measurement: | | |
| | | | 0.8 cm | | |
| Month 2 | Measurement: | Measurement: | Measurement: | Site: Bone | Total Sum = |
| Dec. 20, 2001 | 2.0 × 2.0 cm | 1.5 × 1.0 cm | 0.5 cm | Method: Bone | 4.0 cm |
| | | | | Scan | % Change = |
| | | | | Bony Mets | −31% |
| Month 3 | Measurement: | Measurement: | Measurement: | Site: Bone | Total Sum = |
| Jan. 22, 2002 | 2.1 × 1.5 cm | 1.5 × 1.2 cm | 0.3 cm | Method: Bone | 3.9 cm |
| | | | | Scan | % Change = |
| | | | | Bony mets | −33% |
| | | | | grossly stable | |
| | | | | compared with | |
| | | | | Nov. 19, 2001 | |
| Month 4 | Measurement: | Measurement: | Measurement: | | Total Sum = |
| Mar. 08, 2002 | 2.0 × 1.5 cm | 2.0 × 2.0 cm | 0.5 cm | | 4.5 cm |
| | | | | | % Change = |
| | | | | | −24% |
| Month 5 | Measurement: | Measurement: | Measurement: | | Total Sum = |
| Apr. 17, 2002 | 3.0 × 2.5 cm | 2.0 × 1.5 cm | 0.5 cm | | 5.5 cm |
| | | | | | % Change = |
| | | | | | −5% |

### Example 4

### Efficacy of BZL101 in Humans

In order to demonstrate the safety and clinical activity of oral BZL101 in combination with anastrozole or fulvestrant, an aqueous extract from Scutellaria Barbata D. Don in combination with anastrozole or fulvestrant is studied in human patients with either advanced or early breast cancer.

Eligible patients have histologically confirmed cancer and measurable disease. Patients do not receive any other chemotherapy, hormone therapy or herbal medicine during the trial. Patients receive 350 ml (dry residue from 180 g BZL; approximately 12 grams dry soluble BZL extract) concentrated BZL101 extract per day in additional to varying doses of a second chemotherapeutic agent until disease progression, toxicity or personal preference caused them to discontinue. The primary endpoints are safety, toxicity and tumor response.

Patients meeting one or more of the following criteria are enrolled:

Advanced (metastatic) breast cancer
Nuclear estrogen receptor (ER) negative—i.e. the cancer expresses ER at a level that does not exceed a predetermined threshold (lower limit)
Nuclear estrogen receptor (ER) positive—i.e. the cancer expresses ER at a level that exceeds a predetermined threshold (lower limit)

Early stage (non-metastatic) breast cancer
Nuclear estrogen receptor (ER) negative—i.e. the cancer expresses ER at a level that does not exceed a predetermined threshold (lower limit)
Nuclear estrogen receptor (ER) positive—i.e. the cancer expresses ER at a level that exceeds a predetermined threshold (lower limit)

ER status (ER⁺ or ER⁻) is determined by accepted methods, e.g. by fluoroscopically or isotopically labeled antibody assay or gene chip analysis. Cancer grade is determined by methods known to the clinical oncologist, such as by histological methods known in the art.

Patients are classified as early stage (i.e. non-metastatic) or advanced (metastatic) and are enrolled and are treated with BZL in combination with anastrozole or fulvestrant.

| ER Status | Cancer Grade | BZL101 | Combo Drug |
|---|---|---|---|
| + | Advanced | 1x | |
| + | Advanced | 2x | |
| + | Advanced | 4x | |
| + | Advanced | 8x | |
| − | Advanced | 1x | |
| − | Advanced | 2x | |
| − | Advanced | 4x | |
| − | Advanced | 8x | |
| + | Advanced | 0 | Anastrozole |
| − | Advanced | 0 | Anastrozole |
| + | Advanced | 0 | Fulvestrant |
| − | Advanced | 0 | Fulvestrant |
| + | Advanced | 1x | Anastrozole |
| + | Advanced | 2x | Anastrozole |
| + | Advanced | 4x | Anastrozole |
| + | Advanced | 8x | Anastrozole |
| − | Advanced | 1x | Anastrozole |
| − | Advanced | 2x | Anastrozole |
| − | Advanced | 4x | Anastrozole |
| − | Advanced | 8x | Anastrozole |
| + | Advanced | 1x | Fulvestrant |
| + | Advanced | 2x | Fulvestrant |
| + | Advanced | 4x | Fulvestrant |
| + | Advanced | 8x | Fulvestrant |
| − | Advanced | 1x | Fulvestrant |
| − | Advanced | 2x | Fulvestrant |
| − | Advanced | 4x | Fulvestrant |
| − | Advanced | 8x | Fulvestrant |
| + | Early | 1x | Anastrozole |
| + | Early | 2x | Anastrozole |
| + | Early | 4x | Anastrozole |
| + | Early | 8x | Anastrozole |
| − | Early | 1x | Anastrozole |
| − | Early | 2x | Anastrozole |
| − | Early | 4x | Anastrozole |
| − | Early | 8x | Anastrozole |
| + | Early | 1x | Fulvestrant |
| + | Early | 2x | Fulvestrant |
| + | Early | 4x | Fulvestrant |
| + | Early | 8x | Fulvestrant |
| − | Early | 1x | Fulvestrant |
| − | Early | 2x | Fulvestrant |
| − | Early | 4x | Fulvestrant |
| − | Early | 8x | Fulvestrant |

“Advanced” tumors are metastatic
“Early” tumors are non-metastatic
Multipliers (1x, 2x, etc.) indicate the amount of BZL101 given. BZL101 is a composition comprising the dry solid residue of an extract of 180 g of Scutellaria barbata D. Don (BZL); 1x indicates that the dry solid residue of an extract of 180 g of BZL is administered per day; thus 2x would be the dry solid residue of 360 g of BZL, and so forth.

Safety monitoring is done on a continuous basis and patients are seen by a physician for examination at baseline at regular intervals. Adverse events are graded using Common Toxicity Criteria version 2, assigned a category by organ system and coded in relation to study drug as remote, possible, probably or definitely related. Baseline tumor assessments are done within 14 days of initiation of study drug and every three months. Responses are assessed using RECIST criteria. Study drugs are administered at every visit, and at this visit compliance and a review of dosages taken is performed. BZL101 extract is provided as a liquid in a sealed and labeled aluminum packet containing a full daily dose that is administered in a split dose twice a day. Daily BZL extract is administered until the determination of tumor progression or dose limiting toxicity is encountered, or until the subject decided to voluntarily discontinue, in which case, the reason for discontinuation is obtained. Additional chemotherapeutic agents, when administered, are administered according to established procedures for the specific drugs. In some instances, some fraction of the minimum effective dose is administered (e.g. about 0.1×to about 0.8×the normal minimum effective dose).

### CONCLUSION

The herbal extract BZL101, its uses for the inhibition of solid tumor cancer cells and the treatment of such cancers in patients are described herein. Although certain embodiments and examples have been used to describe the present invention, it will be apparent to those skilled in the art that changes to the embodiments and examples may be made without departing from the scope and spirit of this invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating cancer, comprising administering to a patient a therapeutically effective amount of first active agent comprising an extract of Scutellaria barbata D. Don in combination with a second active agent.

2. The method of claim 1, wherein the extract of Scutellaria barbata D. Don comprises a hot water or ethanolic extract of about 1 to about 20,000 g of Scutellaria barbata D. Don.

3. The method of claim 1, wherein the extract of Scutellaria barbasta D. Don comprises a hot water or ethanolic extract of about 500 to about 10,000 g of Scutellaria barbata D. Don.

4. The method of claim 1, wherein the combination of the first active agent and the second active agents produces a synergistic effect.

5. The method of claim 1, wherein the extract of Scutellaria barbata and the second active agent are formulated in the same dosage form.

6. The method of claim 1, wherein the extract of Scutellaria barbata is administered before, after, or simultaneously with the second active.

7. The method of claim 1, wherein the second agent is: an aromatase inhibitor, an androgen antagonist, a gonadotropin releasing hormone agonist, an estrogen receptor antagonist, an EGF receptor tyrosine kinase antagonist, a VEGF receptor tyrosine kinase antagonist, a RET tyrosine kinase activity antagonist, an endothelin A receptor antagonist, a Src kinase antagonist, an Abl kinase antagonist, a CDK antagonist, a MEK 1 antagonist, a MEK 2 antagonist, an aurora kinase antagonist, or combinations thereof.

8. The method of claim 1, wherein the second agent is anastrozole, bicalutamide, goserelin, fulvestrant, gefitinib, vandetanib, AZD2171, AZD4054, AZD0530, AZD5438,AZD 6244,AZD 1152, or combinations thereof.

9. The method of claim 1, wherein the cancer is breast cancer, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

10. The method of claim 1, wherein the cancer is breast cancer.

11. The method of claim 1, wherein the cancer is ERα- and/or ERβ-breast cancer.

12. The method of claim 1, wherein further comprising a third active agent.

13. The method of claim 1, further comprising an alkylating agent; an antimetabolite; a plant alkaloid, a vinca alkaloid, a podophyllotoxin, a coichicine derivative, a taxane; a cytotoxic antibiotic; a platinum compound; a methylhydrazine; a monoclonal antibody, or combinations thereof.

14. A kit for the treatment of cancer comprising a pharmaceutically acceptable amount of a first active agent comprising an extract of Scutellaria barbata D. Don and a second active agent selected from: aromatase inhibitor, an androgen antagonizing agent, an agonist of gonadotropin releasing hormone, an estrogen receptor antagonist, a tyrosine kinase inhibitor, an endothelin A receptor antagonist, an Src kinase inhibitor, an Abl kinase inhibitor, a CDK inhibitor, an inhibitor of MEK 1, MEK 2 or both, and an aurora kinase inhibitor.

15. The kit of claim 14, wherein the second active agent is an EGF receptor tyrosine kinase inhibitor, a VEGF receptor tyrosine kinase inhibitor, an RET tyrosine kinase inhibitor.

16. The kit of claim 14, wherein the second active agent is: anastrozole, bicalutamide, goserelin, fulvestrant, gefitinib, vandetanib, AZD2171, AZD4054, AZD0530, AZD5438,AZD 6244,AZD 1152, or combinations thereof.

17. The kit of claim 14, further comprising a third active agent.

18. The kit of claim 14, wherein the third active agent is an EGF receptor tyrosine kinase inhibitor, a VEGF receptor tyrosine kinase inhibitor, an RET tyrosine kinase inhibitor.

19. The kit of claim 14, wherein the third active agent is: anastrozole, bicalutamide, goserelin, fulvestrant, gefitinib, vandetanib, AZD2171, AZD4054, AZD0530, AZD5438,AZD 6244,AZD 1152, or combinations thereof.
